(19) United States
(12) Patent Application Publication
(10) Pub. No.: US 2019/0094204 A1
(43) Pub. Date: Mar. 28, 2019

(54) MEMBRANE TRANSPORTER ASSAY AND METHODS OF USE

(71) Applicant: Howard Hughes Medical Institute, Chevy Chase, MD (US)

(72) Inventor: Jacob Keller, Chevy Chase, MD (US)

(21) Appl. No.: 16/138,249

(22) Filed: Sep. 21, 2018

Related U.S. Application Data
(60) Provisional application No. 62/562,026, filed on Sep. 22, 2017.

(51) Int. Cl. G01N 33/487; G01N 33/94; G01N 33/58; C12M 1/34; G01N 21/64; G01N 33/68; G01N 15/14; C09B 69/06

(52) U.S. Cl. CPC G01N 33/48728; G01N 33/942; G01N 33/582; C12M 1/34; G01N 2015/1006; G01N 33/68; G01N 15/1475; C09B 69/06; G01N 33/48721; G01N 21/64

(57) ABSTRACT

This document provides materials and methods for a membrane transporter assay (e.g., an oscillating stimulus transporter assay (OSTA)). OSTA can be used to obtain temporal readouts of transporter activity and to screen for membrane transporter modulators (e.g., agonists or antagonists).

### CROSS REFERENCE TO RELATED APPLICATIONS

This application claims benefit under 35 U.S.C. § 119(e) to U.S. Application No. 62/562,026 filed on Sep. 22, 2017.

### BACKGROUND

### 1. Technical Field

This document relates to materials and methods for a membrane transporter assay (e.g., an oscillating stimulus transporter assay (OSTA)). OSTA can be used to obtain temporal readouts of transporter activity. For example, OSTA can be used to screen for membrane transporter modulators (e.g., agonists or antagonists) having particular pathophysiological interest. This document also provides materials and methods for modulating membrane transporter function. For example, modulators of membrane transporters (e.g., drugs and other stimuli) can be used to treat membrane transporter associated diseases and/or disorders (e.g., cystic fibrosis, stroke, epilepsy, Alzheimer's disease, Huntington's disease, and amyotrophic lateral sclerosis).

### 2. Background Information

Membrane transporter proteins enable the passage of nearly all biologically relevant molecules across the otherwise-impermeable lipid membranes of cells and organelles, and as such, are of great importance to life. Genes encoding membrane transporter proteins constitute >10% of the human genome, and mutations thereof are implicated in a wide range of human genetic diseases and cancers (Sahoo et al., 2014 Front. Physiol. 5:91). Despite this importance, methods for functional measurement have been limited. Transporter proteins are embedded in lipid membranes making purification and characterization of active transporters difficult or even impossible.

### SUMMARY

This document provides materials and methods for a membrane transporter assay (e.g., an oscillating stimulus transporter assay (OSTA)). OSTAs can be used to obtain readouts of transporter activity such as temporal readouts. In some cases, OSTAs can be used to screen for modulators of a membrane transporter. This document also provides materials and methods for modulating membrane transporter function. For example, modulators (e.g., agonists or antagonists) of membrane transporter can be used to treat membrane transporter associated diseases and/or disorders (e.g., cystic fibrosis, stroke, epilepsy, Alzheimer's disease, Huntington's disease, amyotrophic lateral sclerosis (ALS)).

As demonstrated herein, OSTA can be used to monitor (e.g., characterize and/or analyze) the function of membrane transporters embedded in cellular membranes of living cells. OSTA can be used to characterize conditions under which a membrane transporter (e.g., SLC26a3 and SLC26a2) transports most effectively. OSTA can be used to characterize membrane transporter modulator kinetics (e.g., inhibition kinetics) and analyze responsiveness of a membrane transporter to one or more membrane transporter modulators. Thus, OSTAs enable facile and rapid measurement of a wide variety of transporter properties.

In general, one aspect of this document features a method of characterizing membrane transporter function (e.g., transport rate, transporter activity, and transporter inhibition). The method includes, or consists essentially of, expressing a sensor in a cell comprising a membrane transporter, wherein the sensor capable of detecting a substrate transported by the membrane transporter; perfusing the cell with a solution including the substrate, wherein said solution changes or oscillates between a low substrate concentration and a high substrate concentration; and detecting the substrate, wherein changes in detection of the substrate can be used to characterize membrane transporter function. The membrane transporter can be an endogenous or exogenous membrane transporter. The membrane transporter can be any transporter for which a sensor exists to detect transporter activity either directly or indirectly, e.g., changes in substrate or co-substrate concentration, or changes in corresponding solution properties such as pH, which depend on transporter activity. Examples of transporters already successfully measured include, but are not limited to the Glut family, the Sglt family, the EAAT family, the SLC26 family, the NBC family, and sodium-alanine co-transporters. These transporter families may specifically include transporters such as glucose transporter 1 (GLUT1), GLUT2, SLC26a3, SLC26a2, Sglt2, EAAT2, or NBCe1-b.

In some aspects, the membrane transporter can be SLC26a3 and the membrane transporter substrate can include Cl— and HCO3-. In some aspects, the membrane transporter can be SLC26a2 and the membrane transporter substrate can include SO42- and OH—. In some aspects, the membrane transporter can be Sglt2 and the membrane transporter substrate can include Na+ and glucose. In some aspects, the membrane transporter can be EAAT2 and the membrane transporter substrate can include glutamate. In some aspects, the membrane transporter can be NBCe1-b and wherein the membrane transporter substrate can include Na+ and HCO3-. In some aspects, the membrane transporter can be Glut family transporters and the membrane transporter substrate can include glucose.

Expressing a sensor can include transfecting the cell with a nucleotide sequence encoding a peptide sensor. The sensor can be a fluorescent sensor. The fluorescent sensor can be a fluorescent Ca2+ sensor (e.g., Furas, OGB, Fluos, or Indos). The fluorescent sensor can be a fluorescent Mg2+ sensor (e.g., mag-Indo, mag-Fura, or mag-Fluo). The fluorescent sensor can be a fluorescent voltage sensor (e.g., di-ANNEPSs or PeTs). The fluorescent sensor can be a fluorescent H+ sensor (e.g., a SNARF dye, such as SNARF-5F-AM, or a BCECF dye. The fluorescent sensor can be a fluorescent glucose sensor (e.g.,“Sweetie” a fluorescent glucose biosensor). The fluorescent sensor can be a fluorescent glutamate sensor (e.g., the iGluSnFR protein). Other sensors may be for chloride, tryptophan, nicotine, alanine, glycine, proline, maltose, maltotriose, pH, nucleotides, cyclic nucleotides, dinucleotides, glutathione, or peptides. The cell can be any type of cell. The cell can be a mammalian cell. The cell can be an adherent cell. The cell can be a HEK-293 cell or an E. coli cell.

Perfusing the cell with a solution including the substrate can be a continuous perfusion. The oscillation period can be about 10-120 seconds. The solution can oscillate between about 0-5 M substrate concentration.

Detecting the substrate can include obtaining one or more images of the cell. A series of images of the cell can be obtained using a microscope. In some aspects, the sensor can be a fluorescent sensor and the microscope can be a fluorescence microscope. The series of images of the cell can be a time-lapse movie. The time-lapse movie can include frame rates of about 1-2 Hz.

In some embodiments, this document features a method of characterizing Cl—/HCO3- exchange. The method includes, or consists essentially of, expressing a fluorescent H+ sensor in a cell comprising a Cl—/HCO3- exchange transporter, wherein the fluorescent H+ sensor comprises a SNARF-5F-AM dye; perfusing the cell with a solution comprising Cl—, wherein said solution oscillates between about 8 mM Cl— and about 158 mM Cl—; and detecting the H+, wherein changes in the H+ can be used to characterize Cl—/HCO3- exchange across a cell membrane.

In some embodiments, this document features a method of characterizing SO42-/OH— exchange. The method includes, or consists essentially of, expressing a fluorescent H+ sensor in a cell comprising a SO42-/OH— exchange transporter, wherein the fluorescent H+ sensor comprises a SNARF-5F-AM dye; perfusing the cell with a solution comprising SO42-, wherein said solution oscillates between about 0 mM SO42- and about 100 mM SO42-; and detecting H+, wherein changes in the H+ can be used to characterize SO42-/OH— exchange.

In some embodiments, this document features a method of characterizing glucose transporter function. The method includes, or consists essentially of, expressing a fluorescent glucose sensor in a cell comprising a glucose transporter, wherein the fluorescent glucose sensor is a cytosolic ratiometric peptide; perfusing the cell with a solution comprising glucose, wherein said solution oscillates between about 0 mM glucose and about 2-100 mM glucose; and detecting the glucose, wherein changes in detection of the glucose can be used to characterize glucose transporter function. The glucose transporter can be a Na+-dependent glucose transporter function, and the perfusing step further can include perfusing the cell with a solution comprising Na+ and/or K⁺.

In some embodiments, this document features a method of characterizing glutamate transporter function. The method includes, or consists essentially of, expressing a fluorescent glutamate sensor in a cell comprising a glutamate transporter, wherein the fluorescent glutamate sensor comprises iGluSnFR; perfusing the cell with a solution comprising glutamate, wherein said solution oscillates between about 0 mM glutamate and about 10 mM glutamate; and detecting the glutamate, wherein changes in detection of the glutamate can be used to characterize glutamate transporter function.

In another aspect, this document features a method of identifying a substrate of a membrane transporter. The method includes, or consists essentially of, expressing a sensor in a cell comprising a membrane transporter, wherein the sensor can detect a candidate substrate; perfusing the cell with a solution comprising the candidate substrate, wherein said solution oscillates between a high candidate substrate concentration and a low candidate substrate concentration; and detecting transport of the candidate substrate, wherein transport of the candidate substrate identifies the candidate substrate as the substrate of the membrane transporter.

In another aspect, this document features a method of screening for modulators of membrane transporter function. The method includes, or consists essentially of, expressing a sensor in a cell comprising a membrane transporter, wherein the sensor can detect a substrate transported by the membrane transporter; perfusing the cell with a solution comprising the substrate, wherein said solution oscillates between a low substrate concentration and a high substrate concentration; providing the cell with a candidate modulator; and detecting transport of the substrate, wherein a change in transport of the substrate identifies the candidate modulator as a modulator of membrane transporter function.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

DESCRIPTION OF THE DRAWINGS

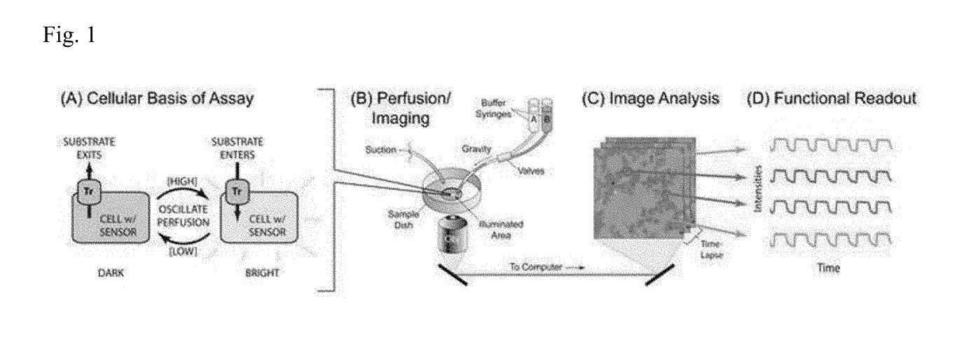

FIG. 1 shows a schematic of an exemplary experimental technique. (A) Cells expressing a transporter of interest and loaded with a fluorescent sensor are perfused with alternating buffers +/− transporter substrate, resulting in oscillating changes in cell fluorescence. (B) Cells are mounted in a culture dish and imaged in an inverted fluorescence microscope while buffers are perfused onto the sample using a simple gravity-fed system switched with computer-controlled valves. (C) Resulting images are stacked for analysis. Typically all processing can be done in ImageJ/FIJI with its large array of built-in and plugin functionalities. (D) After processing, functional readouts are output and plotted as desired.

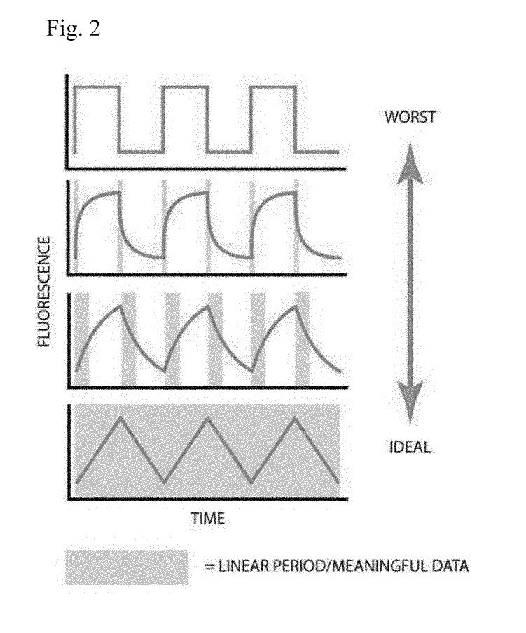

FIG. 2 shows optimal trace parameters for an exemplary OSTA. In the top trace, the response (slope) is too fast to be measured, and therefore provides little or no information. As experimental parameters are optimized (see Examples), curves approaching the triangular waveform at bottom are attained, ideally providing a continuous readout of transport.

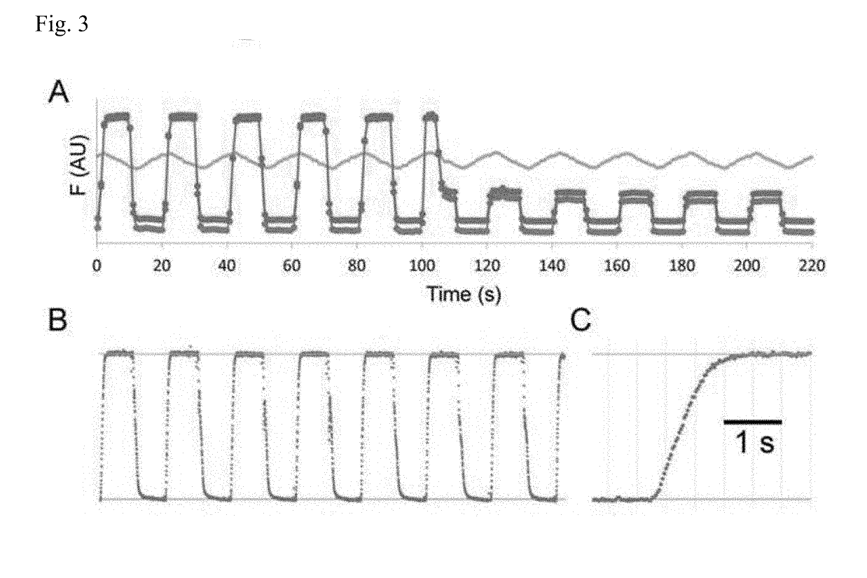

FIG. 3 shows an evaluation of buffer exchange times. (A) Buffer exchanges in the presence of SNARF-5F-loaded, SLC26a3-expressing cells were measured by addition of a fluorescent dye (SNARF-5F) into one of the oscillating buffers used in measuring SLC26a3 function. Fluorescence intensities from a cell-free ROI (raw fluorescence plotted as red and blue traces, one for each emission wavelength) are indicative of presence of dye in perfusion buffer. A cell-containing ROI (gray trace, pH ratios are plotted) was used to measure simultaneously the function of SLC26a3. Units are arbitrary, and detector gain was adjusted to preclude pixel overloads. Frame rate was 2 Hz. At t=110 seconds, syringes were refilled/diluted with normal buffers, resulting in lowered intensities for the dye-containing frames. Note that measurement of transporter activity is indifferent to the buffer/dye changes. (B) Faster frame rates from a line-scan (˜36 scans/sec) of a cell-free region were used to quantify more precisely the time of buffer exchange, using the same buffer protocol as in (A). Note repeatability of measurements and rapidity of exchange. Only one wavelength was measured this time, and green and red lines at top and bottom are to guide the eye. (C) Expanded region of (B) to make time scale more visible. Note almost complete buffer exchange is accomplished in ˜1 second.

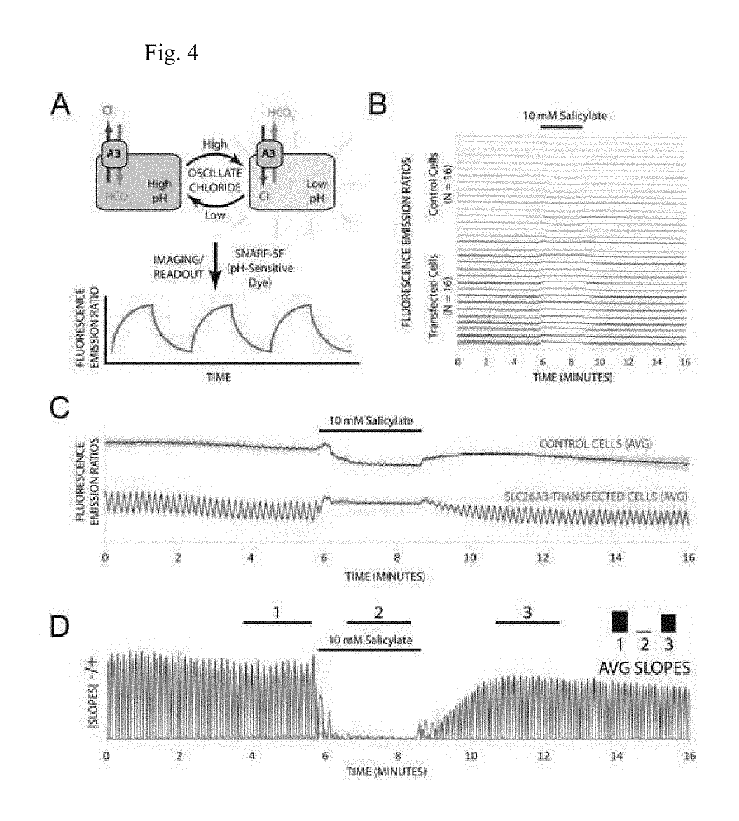

FIG. 4 shows SLC26a3-mediated chloride/bicarbonate exchange is inhibited by 10 mM salicylate. (A) Cells expressing SLC26a3 and loaded with SNARF-5F-AM ratiometric pH indicator dye are perfused with buffers oscillating between 8 and 158 mM chloride in the presence and absence of 10 mM sodium salicylate. Sodium gluconate was substituted for sodium chloride. The signal is the ratio between two emission bands excited with one wavelength (Ex 514 nm, Em 530-595, 600-735 nm; objective Plan-Apochromat 20×/0.8 NA, imaging rate ˜2 Hz). (B) Ratiometric traces from individual cells in one experiment. Traces are vertically shifted by regular intervals for clarity of presentation. Note both abrogation of oscillations as well as absolute pH shift upon exposure to salicylate. Also note salicylate-induced absolute pH shift polarity is opposite between transfected and control cells. (C) Averaged traces from (B), magnified and with error bars indicating standard deviations. Traces are shifted vertically for clarity. (D) Absolute values of slopes of traces in (C), with sign of slope represented by color as indicated at left. Traces from control cells are shown in orange and cyan, and transfected cells are shown as red and blue. Slopes were calculated from a moving window of seven data points (˜3.5 seconds). Inset bar plot shows relative magnitude of slopes in the regions indicated, with control cells omitted for clarity.

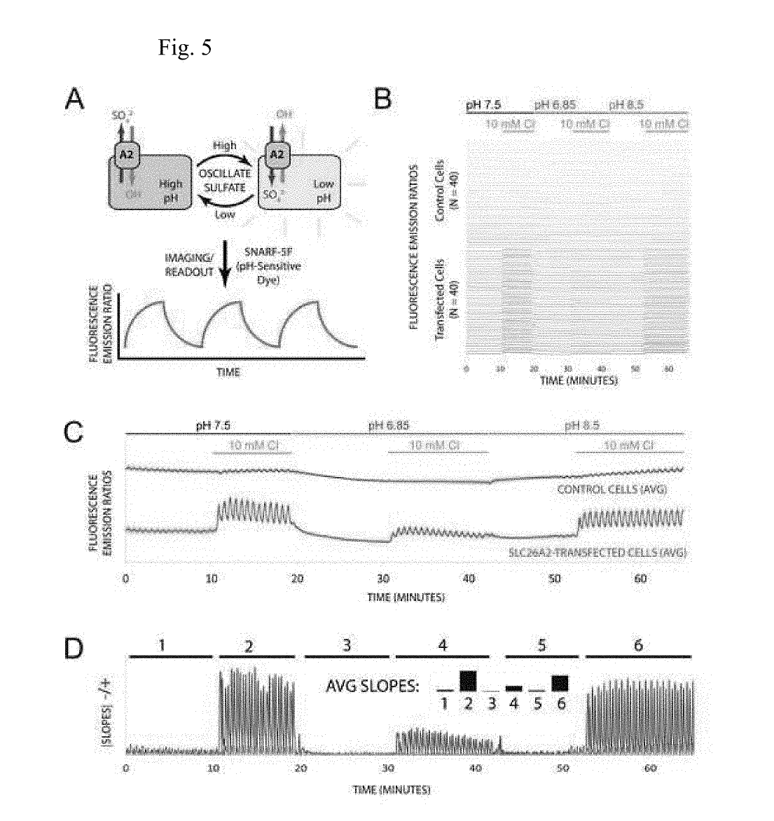

FIG. 5 shows rates of SLC26A2-mediated OH⁻/SO₄²⁻ exchange under various conditions. (A) Cells expressing SLC26a2 and loaded with SNARF-5F-AM ratiometric pH indicator dye are perfused with buffers oscillating between 0 and 100 mM sulfate in the presence and absence of 10 mM chloride, and at three different pH values. Sodium succinate was substituted for sodium sulfate. The signal is the ratio between two emission bands excited with one wavelength (Ex 514 nm, Em 575-595, 595-735 nm; objective EC Plan-Neofluar 10×/0.3 NA M27, imaging rate ˜2 Hz). (B) Individual traces of pH ratios under oscillating 0-100 SO42- derived from 40 transfected and 40 untransfected cells in one field of view, shifted vertically by regular intervals for visualization. pH of solutions as indicated at top, with 10 mM added as indicated. (C) Averaged traces from (B) magnified and with error bars indicating standard deviations. Traces are shifted vertically for clarity. (D) Absolute values of slopes of traces in (C), with sign of slope represented by color as indicated at left. Traces from control cells are shown in green and maroon, and transfected cells are shown as red and blue. Slopes were calculated from a moving window of seven data points (˜3.5 seconds). Inset bar plot shows relative magnitude of slopes in the regions indicated, with control cells omitted for clarity.

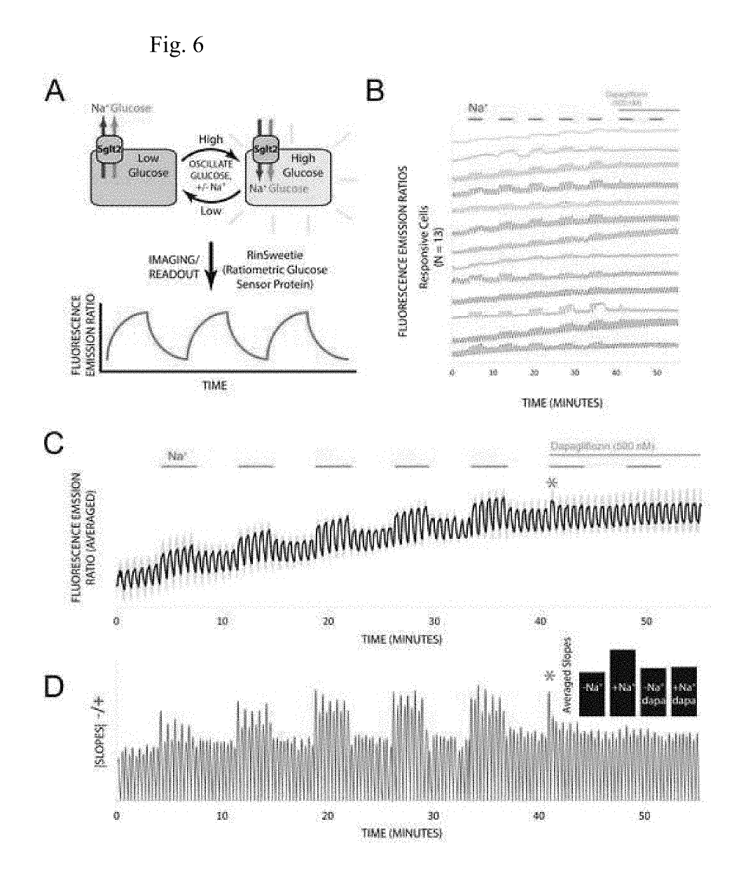

FIG. 6 shows sodium-dependent Sglt2-mediated glucose fluxes and inhibition by dapagliflozin. (A) Cells expressing the sodium-dependent glucose transporter Sglt2 and RinSweetie (an intracellular ratiometric fluorescent glucose sensor protein; Keller et al, manuscript in preparation), are perfused with buffers oscillating between 0 and 2 mM glucose in the presence and absence of 175 mM sodium. Potassium chloride was substituted for sodium chloride (see Example 3). The signal is the ratio between two emission bands excited with one wavelength (Ex 488 nm, Em 505-550, 575-735 nm; objective EC Plan-Neofluar 10×/0.3 NA M27, imaging rate ˜2 Hz). (B) Individual traces of sodium/glucose responsive cells. Dapagliflozin, a potent and specific Sglt2inhibitor, was superadded (500 nM) to perfusion buffers at ˜42 minutes, as indicated. Upwards drift is due to photobleaching of red component of ratiometric sensor. Variability of drift is due to variable kinetics of photobleaching observed in the 575-735 nm channel (not shown). (C) Averaged traces from (B). Asterisk indicates transient response due to dapagliflozin equilibration into perfusion tubing, thereafter promptly squelched by dapaglifozin inhibition. Shading indicates standard deviation between traces after normalization (each trace was simply divided by its time-averaged value). (D) Absolute values of slopes of response in (C), with polarity indicated by color. Inset: bar plot of responses +/− sodium and +/− dapagliflozin.

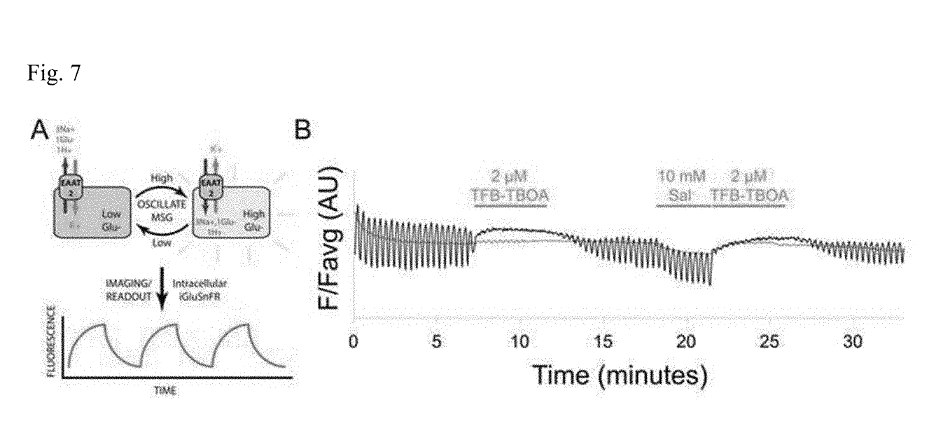

FIG. 7 shows inhibition of EAAT2 by TFB-TBOA and indifference to salicylate. (A) Cells expressing the sodium-dependent glutamate transporter EAAT2 and intracellular iGluSnFR (fluorescent glutamate sensor protein (Marvin et al., 2013 Nat. Methods 10:162-170)), are perfused with buffers oscillating between 0 and 10 mM monosodium glutamate (“MSG”). The signal is emission intensity at one wavelength (Ex 488 nm, Em 505-550; objective EC Plan-Neofluar 10×/0.3 NA M27,imaging rate ˜2 Hz). (B) Trace of glutamate response in the presence of the EAAT2-specific inhibitor TFB-TBOA (2 μM) and sodium salicylate (10 mM), as indicated. Black trace represents EAAT2-positive response, and grey represents EAAT2-negative. Note complete abrogation of oscillating signal by TFB-TBOA and relative indifference to salicylate. Downward tonic response to salicylate in both traces is likely due to salicylate's known action as a protonophore, which lowers intracellular pH and diminishes iGluSnFR intensity (in accordance with iGluSnFR's known pH sensitivity (Marvin et al., 2013 Nat. Methods 10:162-170)).

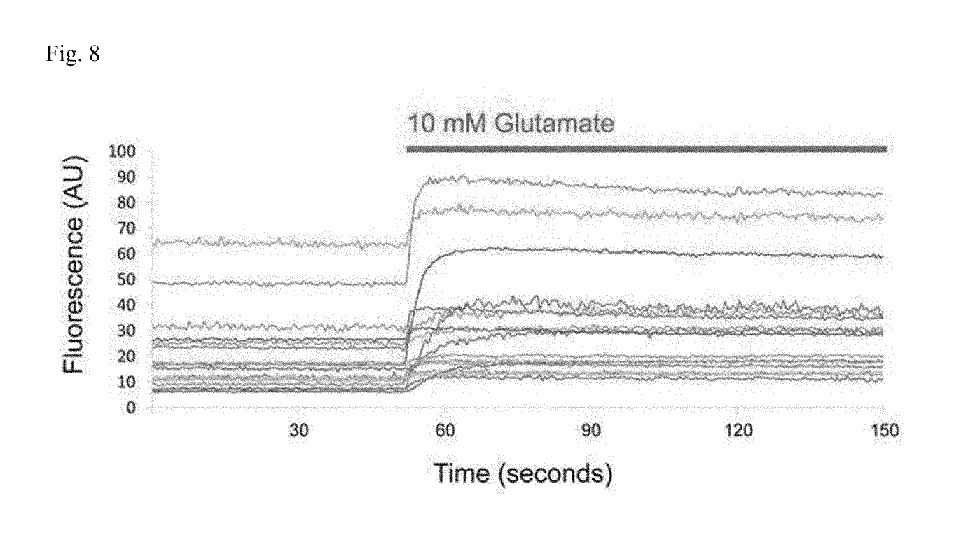

FIG. 8 shows response of glutamate-deprived cells to glutamate. Cells co-transfected with EAAT2 and iGluSnFR incubated for several hours in glutamate-free medium showed a single large step in fluorescence when exposed to glutamate, but were not responsive to subsequent short-term glutamate fluctuations.

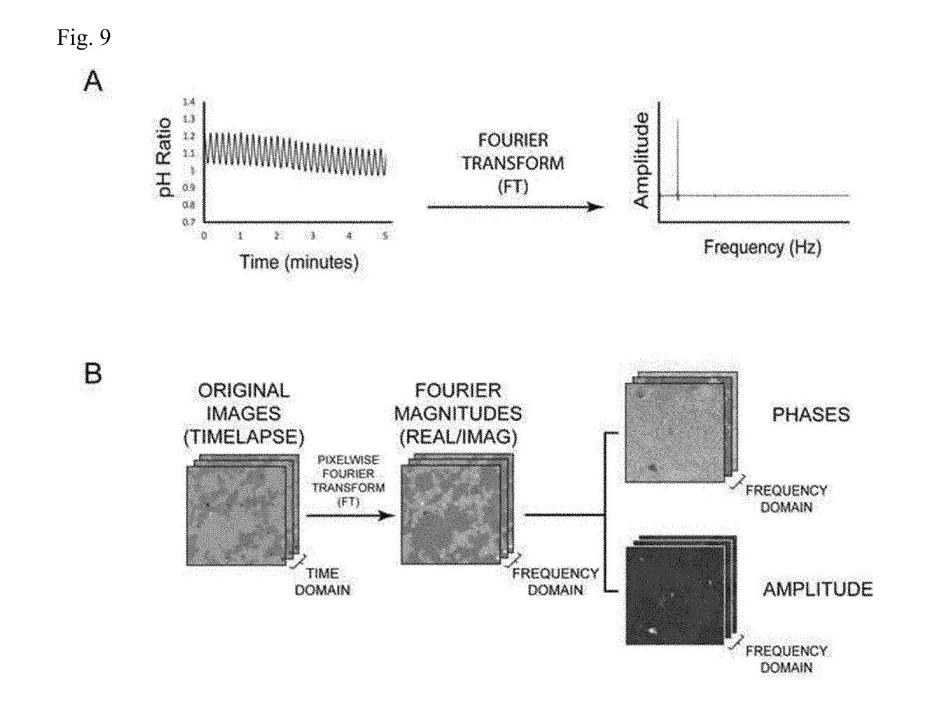

FIG. 9 shows a schematic of FT technique. (A) The FT of a one-dimensional signal in time yields its one-dimensional spectrum in frequency domain. Here, an obviously periodic experimental signal is transformed into a frequency spectrum with one major peak. (B) When the same one-dimensional temporal FT process as in (A) is carried out iteratively for each pixel in a temporal image stack (time-lapse movie), a stack of images in frequency domain can be generated, with each image representing a unique frequency, and with real and imaginary components interleaved. This image stack can in turn be further processed to yield frequency-domain stacks corresponding to either amplitudes or phases.

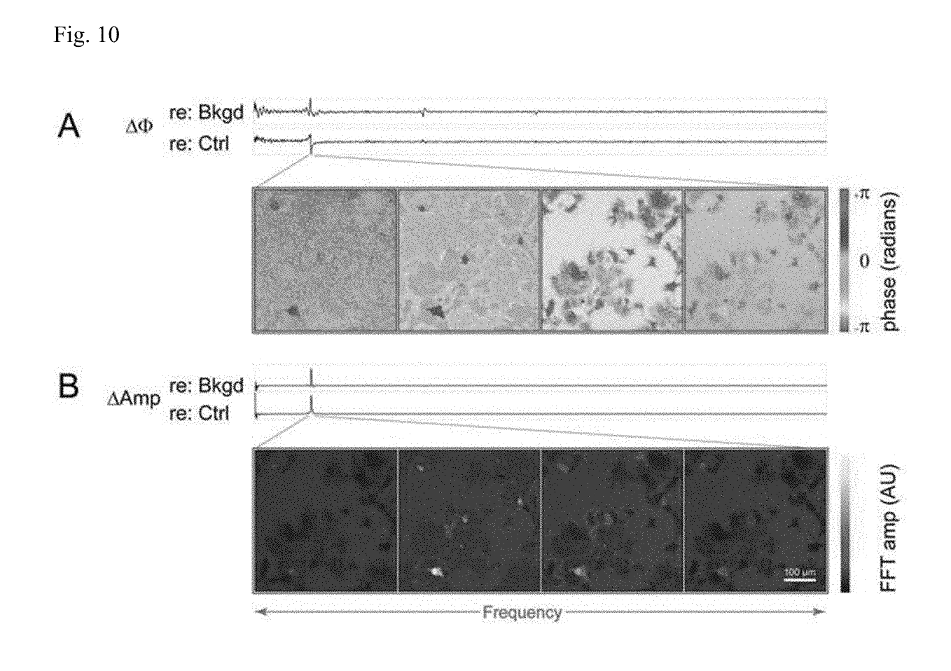

FIG. 10 shows FT phase and amplitude versus frequency in an OSTA time-lapse dataset. (A) FT phase versus frequency. Top: plots of phase differences between an ROI of a transfected cell versus a non-cell ROI (“Bkgd”) or versus a non-transfected cell ROI (“Ctrl”) as a function of frequency. Bottom: images colored by pixel-wise phases at four consecutive frequencies in the transform indicating the efficacy of frequency-specific phase for distinguishing between responsive and control cells. The corresponding calculated periods were 20.58, 20.40, 20.22, and 20.05 seconds (left to right), and stimulus period was set to be 20 seconds. Source of slight discrepancy is unknown. (B) FT amplitude versus frequency. Top: plots of amplitude differences between an ROI of a transfected cell versus a non-cell ROI (“Bkgd”) or versus a non-transfected cell ROI (“Ctrl”) as a function of frequency. Bottom: images colored by pixel-wise amplitudes at four consecutive frequencies in the transform (periods as in (A)), indicating the efficacy of frequency-specific amplitude for distinguishing between transfected and control cells. Amplitude appears to contain more signal than phase under these conditions.

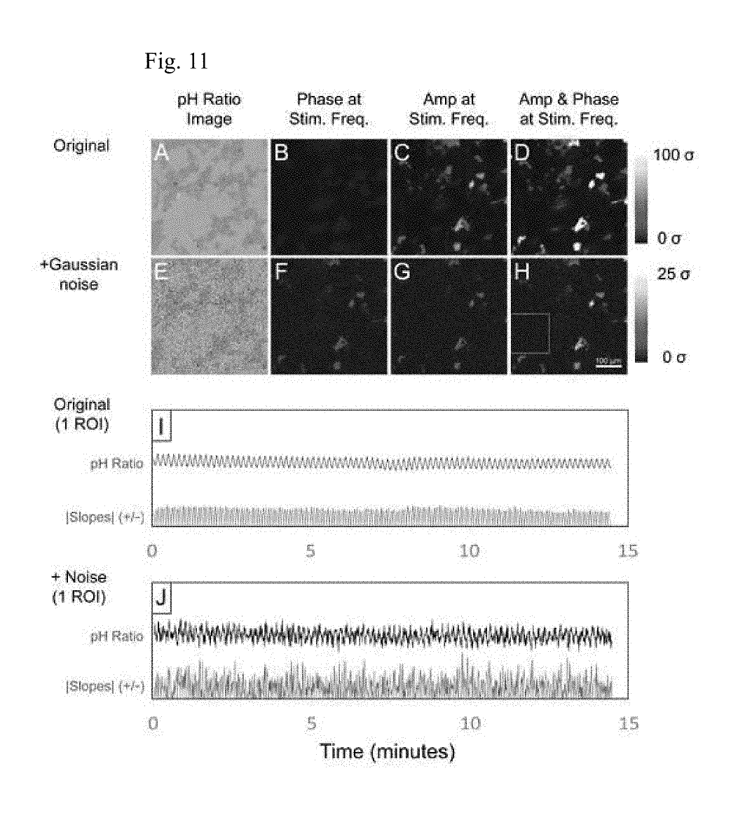

FIG. 11 shows efficacy of frequency-domain analysis in signal retrieval from noisy datasets. (A) Example single unprocessed image from pH ratio time-lapse movie. Cells are clearly distinguishable from background. (B) Image of pixel-wise phase differences, at stimulus frequency, relative to a reference transfected cell; colored by standard deviations derived from a background region (scale at far right, region shown as box in (H)—see Example 4). Contrast is low to allow for comparison to larger signals in (C-D). (C) Image of pixel-wise amplitudes at stimulus frequency, colored by standard deviations with the same scale as (B). (D) Image of combined phase-difference and amplitude information, colored by standard deviations similarly to (B-C). (E) Example single image from pH ratio time-lapse as in (A), set to same contrast range as (A), which tends to enhance visibility of cells. Cells are invisible when contrast range is reset to accommodate all pixel values. (F) Image of pixel-wise phase differences, at stimulus frequency, relative to a reference transfected cell; colored by standard deviations derived from a background region (scale at far right, region shown as box in (H)). Note relative insensitivity of phase information to noise. Note difference in scale compared to BD. (G) Image of pixel-wise amplitudes at stimulus frequency, colored by standard deviations with the same scale as (F). (H) Image of combined phase-difference and amplitude information, colored by standard deviations similarly to (F-G). Inset rectangle indicates ROI used to compute background standard deviations. (I-J) Analogous time-domain analysis of one ROI from the same image series, with and without added noise. (I) Black trace indicates pH ratios from one cell, and the red/blue traces below indicate positive/negative slope magnitudes thereof. (J) Similar to (I) but with the same artificially added noise as in (E-H).

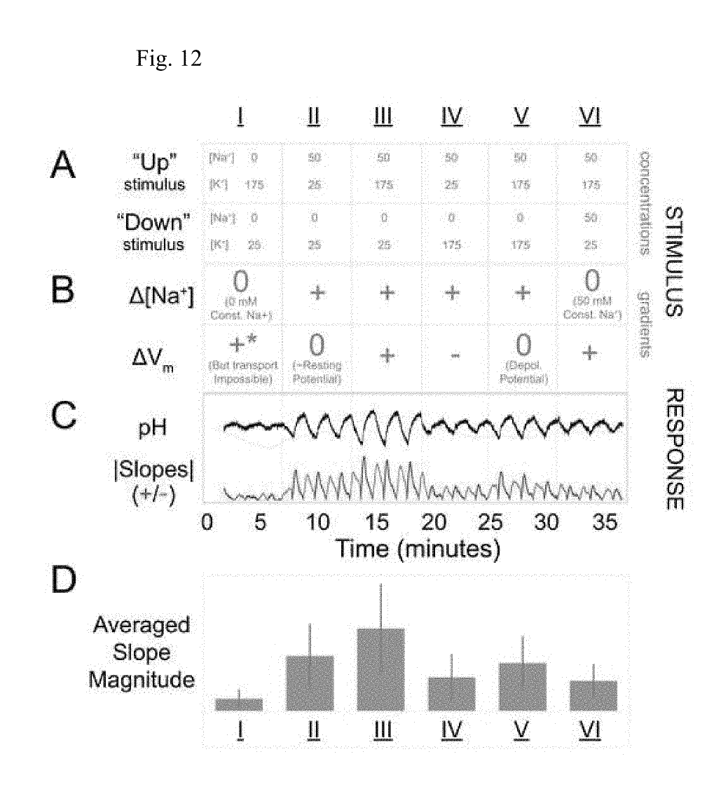

FIG. 12 shows a case study of FT-based de-noising demonstrating electrogenicity and sodium dependence of NBC1e-b-mediated Na⁺/HCO3⁺ transport. (A) Concentrations of ions used in various stages (stages are indicated by roman numerals, top) of the experiment. “Up” and “down” stimulus refer to the direction of pH response in (C). Na⁺ drives flux directly through co-transport, whereas K⁺ affects transport through its alteration of membrane potential. (B) Gradients of Na⁺ ions or K⁺-induced voltage at various stages of experiment. “0” indicates no gradient, and “+” and “−” are arbitrary signs to show whether the gradients promote or oppose transport, based on previously reported transporter properties. (C) Averaged background-subtracted pH response of FT-identified transfected cells during various stages (black trace) with slope magnitudes thereof shown in red/blue for positive/negative values, respectively. Note magnitudes of changes are clearly greatest when the Na⁺ and electrical gradients are in the same direction, although either gradient alone is sufficient to drive transport. Gray trace represents subtracted moving-window background (see text). (D) Bar plots of averaged slope magnitudes during different stages. Error bars represent +/− standard deviation.

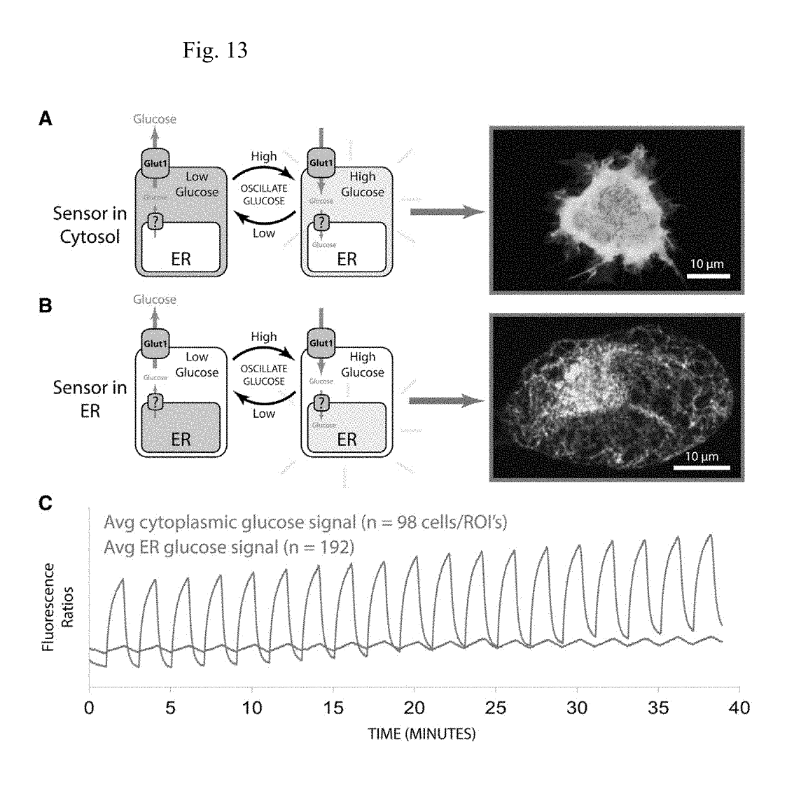

FIG. 13 shows a case study of Glucose Transport into the endoplasmic reticulum (ER) through the measurement of relative glucose fluxes into cytosol and endoplasmic reticulum. (A) Schematic of glucose transport into the cytosol. Sensor is expressed in cytosol and cells are exposed to buffers alternating between 100 mM glucose and sorbitol (inert substitute); the glucose transporter Glut1 was expressed to enhance fluxes. A sample high-magnification image is shown at right (Ex 488/561 nm, Em 495-575/575-617 nm; objective Plan-Apochromat 63×/1.40 NA Oil DIC.) (B) Schematic diagram of assay for ER glucose. All parameters are the same as in (A), but the sensor in this case is expressed in the ER lumen. A sample high-magnification image is shown at right (same microscopic parameters as in A). (C) Quantification of glucose fluxes into cytosol and ER (fluorescence ratios of glucose sensor). The ER signal is smaller but still measureable and linear, indicating possibilities for OSTA-based measurement of transport even in subcellular organelles (Ex 488/561 nm, Em 495-575/575-617 nm; objective EC Plan-Neofluar 10×/0.30; stimulus period 120 s.)

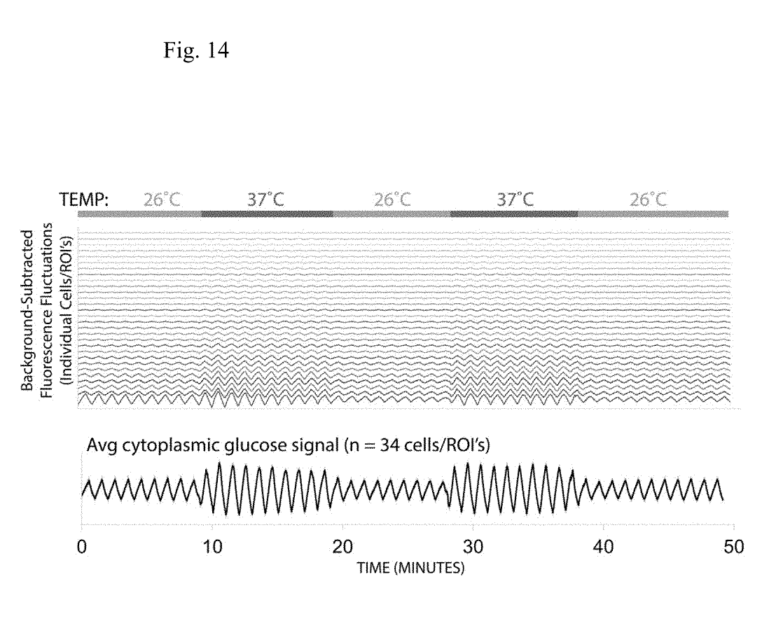

FIG. 14 shows a case study of the effects of temperature changes on Glut2-mediated glucose transport into cytosol. Top: individual traces of moving-window background-subtracted images are shown, with temperature changes of perfusate shown above. Bottom: average of normalized traces is shown. Note barely visible error bars in red indicating 1 SD. Note also increased amplitude at higher temperature. (Ex 488/561 nm, Em 495-575/575-617 nm; objective EC Plan-Neofluar 10×/0.30 NA; stimulus period 60 s.

### DETAILED DESCRIPTION

This document provides materials and methods for a membrane transporter assay (e.g., an oscillating stimulus transporter assay (OSTA)). The membrane transporter assay described herein can be performed on membrane transporters in situ (e.g., embedded in the cellular membrane of living cells). A membrane transporter assay described herein can drive substrate transport and image the response of a sensor to measure transporter function. Further, not only are the methods provided herein are easier and faster both to implement and to run than current methods of studying membrane transporters, but functional imaging techniques provided herein achieve greater temporal resolution and statistical robustness. In some cases, a membrane transporter assay described herein can be used to monitor membrane transporter function (e.g., to monitor membrane transporter function as a function of time). For example, a membrane transporter assay described herein can be used to characterize and/or analyze membrane transporter function as a function of time. For example, OSTA can be used to obtain temporal readouts of transporter activity or to measure time-dependent responses to membrane transporter modulators (e.g., drugs and other stimuli). In some cases, OSTA can be used to screen for membrane transporter modulators (e.g., agonists or antagonists) having particular pathophysiological interest. This document also provides materials and methods for modulating membrane transporter function. For example, modulators of membrane transporter (e.g., drugs and other stimuli) can be used to treat membrane transporter associated diseases and/or disorders (e.g., cystic fibrosis, cerebral stroke, epilepsy, Alzheimer's disease, Huntington's disease, amyotrophic lateral sclerosis (ALS)).

A membrane transporter assay described herein can characterize and/or analyze membrane transporter function in any appropriate cell. A cell can have an endogenous membrane transporter or an exogenous membrane transporter. In cases where a cell has an exogenous membrane transporter, the membrane transporter can be a recombinant membrane transporter. A cell can be an adherent cell or a non-adherent cell. A cell can be from any appropriate source. For example, a cell that can be used in a membrane transporter assay described herein can from a mammal (e.g., human, mouse, hamster, dog, rabbit, primate, and cat), non-mammalian animal, a plant, a bacterium, a fungus (e.g., a yeast), and a protist (e.g., algae). A cell can be any appropriate cell type (e.g., a kidney cell, a neuron, a myocyte, an epithelial cell, a blood cell, and a liver cell). Examples of cells that can be used in a membrane transporter assay described herein include, without limitation, HEK-293, MDCKII, and Caco-2 cells. For example, a cell that can be used in a membrane transporter assay described herein to characterize and/or analyze membrane transporter function can be a HEK-293 cell.

A membrane transporter assay described herein can characterize and/or analyze any appropriate membrane transporter. A membrane transporter can be any membrane protein involved in the movement of a substrate across a lipid bilayer, a cell membrane and/or an organelle membrane. Transport across a cell membrane can be transport from the intracellular side of the cell membrane to the extracellular side of the cell membrane or from the extracellular side of the cell membrane to the intracellular side of the cell membrane. A membrane transporter can facilitate movement of a substrate across a cell membrane by passive transport (e.g., facilitated diffusion) or by active transport (e.g., transport of a substrate across a membrane against its concentration gradient). Active transport can be primary active transport (e.g., requiring chemical energy such as ATP) or secondary active transport (e.g., requiring an electrochemical gradient). A membrane transporter can be a carrier (e.g., allowing only a small amount of substrate to be transported) or a channel (e.g., allowing thousands to millions of substrate molecules, such as ions, to be transported). A membrane transporter can be a glutamate transporter (e.g., an excitatory amino acid transporter (EAAT) or a vesicular glutamate transporter (VGLUT)), an ion transporter (e.g., calcium (Ca2+) pumps, magnesium (Mg2+) transporters, sodium (Na+) transporters, and potassium (K+) transporters), a bicarbonate (HCO3-) transporter, or a sulfate (SO42-) transporter. In some cases, a membrane transporter can be an exchanger (e.g., an anion exchanger) that transports a first substrate (e.g., a first molecule or ion) and exchanges it for a second substrate (e.g., a second molecule or ion). A membrane transporter can be a recombinant membrane transporter.

A membrane transporter can be any of:

1: Channels/pores
α-helical protein channels such as voltage-gated ion channel (VIC), ligand-gated ion channels(LGICs)
β-barrel porins such as aquaporin
channel-forming toxins, including colicins, diphtheria toxin, and others
Nonribosomally synthesized channels such as gramicidin
Holins; which function in export of enzymes that digest bacterial cell walls in an early step of cell lysis.
Pores, which are continuously open to these both environment, because they do not undergo conformational changes. They are always open and active.

2: Electrochemical potential-driven transporters
2.A: Porters (uniporters, symporters, antiporters)
   Glucose transporter
   Monoamine transporters, including:
      Dopamine transporter (DAT)
      Norepinephrine transporter (NET)
      Serotonin transporter (SERT)
      Vesicular monoamine transporters (VMAT)
   Adenine nucleotide translocator (ANT)
2.B: Nonribosomally synthesized porters, such as
   The Nigericin (Nigericin) Family
   The Ionomycin (Ionomycin) Family
2.C: Ion-gradient-driven energizers

3: Primary active transporters
3.A: P-P-bond-hydrolysis-driven transporters:
   ATP-binding cassette transporter (ABC transporter), such as MDR, CFTR
   V-type ATPase; (“V” related to vacuolar).
   P-type ATPase; (“P” related to phosphorylation), such as:
      Na⁺/K⁺-ATPase
      Plasma membrane Ca²⁺ ATPase
      Proton pump
   F-type ATPase; (“F” related to factor), including: mitochondrial ATP synthase, chloroplast ATP synthase
3.B: Decarboxylation-driven transporters
3.C: Methyltransfer-driven transporters
3.D: Oxidoreduction-driven transporters
3.E: Light absorption-driven transporters, such as rhodopsin

4: Group translocators

The group translocators provide a special mechanism for the phosphorylation of sugars as they are transported into bacteria (PEP group translocation)

5: Electron carriers

The transmembrane electron transfer carriers in the membrane include two-electron carriers, such as the disulfide bond oxidoreductases (DsbB and DsbD in E. coli) as well as one-electron carriers such as NADPH oxidase. Often these redox proteins are not considered transport proteins.

A membrane transporter can be a member of a solute carrier family (SLC). A membrane transporter can be a glucose transporter (GLUT). Examples of membrane transporters that can be characterized and/or analyzed with a membrane transporter assay described herein include, without limitation, EAAT1 (SLC1A3), EAAT2 (GLT-1 or SLC1A2), EAAT3 (SLC1A1), EAAT4 (SLC1A6), EAAT5 (SLC1A7), VGLUT1 (SLC17A7), VGLUT2 (SLC17A6), VGLUT3 (SLC17A8), GLUT1 (SLC2A1), GLUT2, SLC26a3, SLC26a2, Sglt2, NBCe1-b, plasma membrane Ca²⁺ ATPase (PMCA), Na⁺/Ca²⁺ exchanger (NCX), MRS2, SLC41 (MgtE), TRPM6/TRPM7, and band 3 anion exchange proteins. In some cases, a membrane transporter can be GLUT1, GLUT2, SLC26a3, SLC26a2, Sglt2, EAAT2, or NBCe1-b. Examples of substrates that can be transported by a membrane transporter include, without limitation, ions (e.g., Ca²⁺, Mg²⁺, Na⁺, H⁺, Zn²⁺, and K⁺), HCO₃⁻, OH⁻, SO₄²⁻, amino acids (e.g., glutamate), small molecules (e.g., glucose), and macromolecules (e.g., proteins, DNA, and RNA). In some cases, a membrane transporter described herein can transport a specific substrate. For example, in cases where the membrane transporter is SLC26a3, the substrate can be Cl⁻ and/or HCO₃⁻. For example, in cases where the membrane transporter is SLC26a2, the substrate can be SO₄²⁻ and/or OH⁻. For example, in cases where the membrane transporter is Sglt2, the substrate can include Na⁺ and/or glucose. For example, in cases where the membrane transporter is EAAT2, the substrate can include glutamate. For example, in cases where the membrane transporter is NBCe1-b, the substrate can include Na⁺ and/or HCO₃⁻.

A membrane transporter assay described herein can use any appropriate sensor. A sensor described herein can detect the presence of substrate. A sensor can indicate the presence of a substrate and may also be referred to herein as an indicator. The sensor can be any sensor capable of detecting a substrate transported by the membrane transporter. In some cases, a sensor can be an intracellular sensor. In some cases, a sensor can detect a specific substrate. For example, a sensor can detect an ion (e.g., Ca²⁺, Mg²⁺, Na⁺, H⁻, or Zn²⁺), HCO₃⁻, OH⁻, SO₄²⁻, an amino acid (e.g., glutamate), a small molecule (e.g., glucose), or a macromolecule (e.g., a protein). In some cases, a substrate can be labeled, and a sensor can detect the label (e.g., chitin binding protein (CBP), maltose binding protein (MBP), glutathione-S-transferase (GST), poly(His) tag, thioredoxin (TRX), poly(NANP), FLAG-tag, V5-tag, Myc-tag, HA-tag, NE-tag, and biotin). A sensor can be any appropriate type of molecule. In some cases, a sensor can be a protein sensor. A sensor described herein can provide an output, such as a visual signal (e.g., fluorescence, colorimetric dyes, absorbance changes, and turbidity) or an electrical signal (e.g., conductivity and resistance). In some cases, a sensor can be a fluorescent sensor. For example, a sensor that can be used to detect the presence of a substrate in a membrane transporter assay described herein can be a fluorescent protein sensor. Examples of sensors that can be used to detect a substrate as described herein include, without limitation, Furas, OGB, Fluos, Indos, mag-Indo, mag-Fura, mag-Fluo, di-ANNEPSs, PeTs, SNARF dyes (e.g., SNARF-5F-AM), BCECF dyes, cytosolic ratiometric peptides, intensity-based glutamate-sensing fluorescent reporters (iGluSnFRs; see, e.g., WO 2013/052946, and Marvin et al., 2013 Nat Methods. 10:162-70). In some cases, a sensor described herein can detect a specific substrate. For example, in cases where the substrate is H⁺/pH, the sensor can be a SNARF or a BCECF. For example, in cases where the substrate is Ca²⁺, the sensor can be a Fura, a OGB, a Fluos, or a Indos. For example, in cases where the substrate is Mg²⁺, the sensor can be a mag-Indo, a mag-Fura, or a mag-Fluo. For example, in cases where the substrate is K⁺ (e.g., to detect transmembrane potential), the sensor can be a di-ANNEPSs or a PeTs. For example, in cases where the substrate is H⁺ (e.g., to detect pH changes associated with changes in, for example, OH⁻ or HCO₃⁻), the sensor can be a SNARF dye (e.g., SNARF-5F-AM) or a BCECF dye. For example, in cases where the substrate is glucose, the sensor can be a cytosolic ratiometric peptide. For example, in cases where the substrate is glutamate, the sensor can be an iGluSnFR. The sensor described herein can be provided to a cell by any appropriate means. For example, a sensor can be provided to a cell by transfection, transduction, or electroporation, into a cell. In some cases, a membrane transporter assay described herein can include expressing a nucleic acid sequence encoding a protein sensor in a cell having a membrane transporter described herein. A nucleic acid sequence encoding a protein sensor can be a recombinant nucleic acid sequence. A nucleic acid sequence encoding a protein sensor can be naked nucleic acid or can be in a vector (e.g., a plasmid vector, an expression vector, or a viral vector). For example, a sensor can be provided to a cell described herein by transfecting the cell with an expression vector including a nucleic acid sequence encoding a protein sensor. In some cases, a membrane transporter assay described herein can include loading a cell having a membrane transporter described herein with a small molecule sensor. For example, a sensor can be provided to a cell described herein by electroporating the cell with a small molecule sensor.

A membrane transporter assay described herein can include providing a cell described herein (e.g., having a membrane transporter and a sensor) with substrate. In some cases, a membrane transporter can be an exchanger (e.g., an anion exchanger), and a membrane transporter assay described herein can include providing a cell having a membrane transporter and a sensor with a first substrate and a second substrate. A substrate can be provided in a solution. In some cases, providing a cell described herein with substrate can include perfusion the cell with a substrate solution. The perfusion can be continuous or discontinuous. In some cases, a cell having a membrane transporter and a sensor with substrate can include continuously perfusion the cell with a substrate solution. A substrate solution can oscillate between having a high substrate concentration and having a low substrate concentration. For example, a solution having a high substrate concentration can have at least about 1 mM substrate (e.g., at least about 2, at least about 10, at least about 25, at least about 50, at least about 75, at least about 100, at least about 120, at least about 130, at least about 140, at least about 150, at least about 160, at least about 170, at least about 180, at least about 190, or at least about 200 mM). For example, a solution having a low substrate concentration can have no greater than about 0 mM substrate (e.g., greater than about 1, greater than about 2, greater than about 5, greater than about 8, greater than about 10, greater than about 15, or greater than about 25 mM). In some cases, a substrate solution can oscillate between about 0 and about 5 M substrate (e.g., can oscillate between about 0 mM and about 150 mM, can oscillate between about 0 mM and about 100 mM, can oscillate between about 0 mM and about 50 mM, can oscillate between about 0 mM and about 10 mM, can oscillate between about 0 mM and about 2 mM, can oscillate between about 5 mM and about 160 mM, can oscillate between about 8 mM and about 158 mM, can oscillate between about 10 mM and about 150 mM, or can oscillate between about 25 mM and about 150 mM substrate). For example, a substrate solution can oscillate between having about 8 mM and about 158 mM Cl⁻. For example, a substrate solution can oscillate between having about 0 mM and about 100 mM SO₄²⁻. For example, a substrate solution can oscillate between having about 0 mM and about 150 mM K⁺. For example, a substrate solution can oscillate between having about 25 mM and about 150 mM K⁺. For example, a substrate solution can oscillate between having about 0 mM and about 2 mM glucose. For example, a substrate solution can oscillate between having about 0 mM and about 10 mM glutamate (e.g., monosodium glutamate). In cases where a first substrate and a second substrate are provided, the first substrate and the second substrate can be oscillated in conjunction or in opposition. An oscillation period is the amount of time it takes to complete a full cycle of perfusion subjecting a cell to both a high substrate concentration and a low substrate concentration. For example, an oscillation period of 10 seconds would include perfusion a cell with a high substrate concentration of 5 seconds and perfusion a cell with a low substrate concentration for 5 seconds. In some cases, an oscillation period can be about 0.1 to about 600 seconds/period (e.g., about 10 to about 120, about 15 to about 100, about 20 to about 75, about 25 to about 50, about 10 to about 15, about 15 to about 20, about 20 to about 25, or about 25 to about 30 seconds/period). For example, an oscillation period can be about 10 seconds/period. Oscillation periods can also be asymmetrical, e.g. high substrate concentration for about 2 seconds and low substrate for about 8 seconds, etc. Substrate gradients created by such oscillations drive membrane transporter function and thus drive transport of the substrate.

In some cases, a membrane transporter assay described herein can include providing a cell described herein (e.g., having a membrane transporter and a sensor) with one or more additional agents that help drive membrane transporter function. Examples of additional agents that can help drive membrane transporter function include, without limitation, adenosine triphosphate (ATP), additional ions (e.g., to create an electrochemical gradient).

A membrane transporter assay described herein can include one or more imaging, voltammetric, absorbance or intensitometric methods. In some cases, a membrane transporter assay described herein can include obtaining images of a cell described herein (e.g., having a membrane transporter and a sensor) during perfusion with a substrate solution oscillating between having a high substrate concentration and having a low substrate concentration. For example, at least one instensometric measurement or image can be obtained during perfusion of a cell described herein with a substrate solution having a high substrate concentration and at least one measurement or image can be obtained during perfusion of a cell described herein with a substrate solution having a low substrate concentration. An image can be used to determine the location of a substrate. For example, in cases where a sensor is an intracellular sensor, detection of the substrate indicates that the substrate is intracellular, and absence of detection of the substrate indicates that the substrate is extracellular. Images can be obtained using any appropriate method. In some cases, images can be obtained using a microscope (e.g., confocal microscope). It will be understood that the method of obtaining on image will depend on the type of sensor. For example, in cases where a sensor is a fluorescent sensor, images can be obtained using a fluorescence microscope (e.g., a confocal fluorescence microscope, an epifluorescence microscope, or an inverted fluorescence microscope). In some cases, a microscopic field used to obtain an image can include a view of more than one cell. For example, a microscopic field of view can include a view of at least 2 cells (e.g., at least 5, at least 8, at least 10, at least 12, at least 15, at least 20, at least 25, at least 30, at least 35, at least 40, at least 45, at least 50, at least 60, at least 70, at least 80, at least 90, at least 100, at least 125, at least 150, at least 175, at least 200, or at least 5000 cells). Membrane transporter function in each cell present in a microscopic field of view can be independently measured, permitting large datasets to be collected simultaneously. In some cases, a microscopic field of view including at least 2 cells can include at least 1 experimental cell (e.g., a transporter-positive cell) and at least one control cell (e.g., a transporter-negative cell).

In some cases, a membrane transporter assay described herein can include obtaining a series (e.g., temporal series) of images of a cell having a membrane transporter and a sensor and perfused with a substrate solution oscillating between having a high substrate concentration and having a low substrate concentration. A series of images can be independently obtained or can be frames isolated from a time-lapse movie. Images obtained from a time-lapse movie can be obtained at fast frame rate (e.g., about 2 Hz). A series of images can be obtained manually or automatically. In some cases, a series of images can be stacked, for example, in temporal series. In some cases, a stacked image can provide a readout of the membrane transporter function. For example, a stack of images can provide one or more measurements of membrane transporter function in a time domain. For example, a stack of images can provide one or more measurements of membrane transporter function in a frequency domain. A stack of images described herein can be evaluated using any appropriate image processing software (e.g., ImageJ, FIJI, and ImageJ/FIJI). In some cases, a stack of images described herein can be processed. For example, a stack of images can be subjected to FT analysis.

This document also provides methods of using a membrane transporter assay described herein. In some cases, a membrane transporter assay described herein can be used to characterize and/or analyze membrane transporter function. Any appropriate membrane transporter function can be assayed using a membrane transporter assay described herein. Examples of membrane transporter functions can be assayed include, without limitation, rate of substrate transport, kinetics of substrate transport, and electrophysiology. A method of characterizing membrane transporter function can include expressing a sensor in a cell having a membrane transporter, perfusing the cell with a solution having substrate transported by the membrane transporter, and detecting the substrate. During the perfusion, the substrate solution can oscillate between having a low substrate concentration and having a high substrate concentration. Changes in detection of the substrate can be used to characterize membrane transporter function. In some cases, a change in substrate location can be used to characterize membrane transporter function. A change from detecting intracellular substrate to detecting extracellular substrate can be indicative of substrate transport. A change from detecting extracellular substrate to detecting intracellular substrate can be indicative of substrate transport. In some cases, a change in amount of substrate transported can be used to characterize transport rate. For example, an increase in the amount of substrate transported can be indicative of an increase in transport rate. For example, a decrease in the amount of substrate transported can be indicative of a decrease in transport rate.

Any membrane transporter assay described herein can be a high-throughput assay. A high-throughput membrane transporter assay described herein can be performed in a multi-well cell culture plate. Examples of multi-well cell culture plates that can be used in a high-throughput membrane transporter assay described herein include, without limitation, 6 well plates, 12 well plates, 24 well plates, 48 well plates, 96 well plates, 384 well plates, and 1536 well plates. In some cases, different wells of a multi-well cell culture plate can have cells having the same membrane transporter. For example, different wells of a multi-well cell culture plate having cells having the same membrane transporter can be treated with different substrates (e.g., different candidate substrates). Alternatively, or in parallel, different wells of a multi-well cell culture plate having cells having the same membrane transporter can be treated with the same substrate, but treated with different modulators.

In some cases, OSTA can be used to characterize Cl⁻/HCO₃⁻ exchange. For example, a method of characterizing Cl⁻/HCO₃⁻ exchange can include expressing a fluorescent pH sensor (e.g., a SNARF-5F-AM dye) in a cell having a Cl⁻/HCO₃⁻ exchanger, perfusing the cell with a solution having Cl⁻, and detecting the Cl⁻. During the perfusion, the Cl⁻ solution can oscillate between about 8 mM Cl⁻ and having about 158 mM Cl⁻. Changes in detection of Cl⁻ can be used to characterize Cl⁻/HCO₃⁻ exchange function.

In some cases, OSTA can be used to characterize SO₄²⁻/OH⁻ exchange. For example, a method of characterizing SO₄²⁻/OH⁻ exchange can include expressing a fluorescent pH sensor (e.g., a SNARF-5F-AM dye) in a cell having a SO₄²⁻/OH⁻ exchanger, perfusing the cell with a solution having SO₄²⁻, and detecting the SO₄²⁻. During the perfusion, the SO₄²⁻ solution can oscillate between about 0 mM SO₄²⁻ and about 100 mM SO₄²⁻. Changes in detection of SO₄²⁻ can be used to characterize SO₄²⁻/OH⁻ exchange.

In some cases, OSTA can be used to characterize a glucose transporter. For example, a method of characterizing a glucose transporter function can include expressing a fluorescent glucose sensor (e.g., a cytosolic ratiometric peptide) in a cell having a glucose transporter, perfusing the cell with a solution having glucose, and detecting the glucose. During the perfusion, the glucose solution can oscillate between about 0 mM glucose and about 2 mM glucose. Changes in detection of glucose can be used to characterize glucose transporter function.

In some cases, OSTA can be used to characterize a Na⁺-dependent glucose transporter. For example, a method of characterizing a Na⁺-dependent glucose transporter function can include expressing a fluorescent glucose sensor (e.g., a cytosolic ratiometric peptide) in a cell having a Na⁺-dependent glucose transporter, perfusing the cell with a solution having glucose and Na⁺, and detecting the glucose. During the perfusion, the glucose solution can oscillate between about 0 mM glucose and about 2 mM glucose. Changes in detection of glucose can be used to characterize Na⁺-dependent glucose transporter function.

In some cases, OSTA can be used to characterize a glutamate transporter. For example, a method of characterizing glutamate transporter function can include expressing a fluorescent glutamate sensor (e.g., iGluSnFR) in a cell having a glutamate transporter, and perfusing the cell with a solution having glutamate (e.g., MSG), and detecting the glutamate. During the perfusion, the glutamate solution can oscillate between about 0 mM glutamate and about 10 mM glutamate. Changes in detection of glutamate can be used to characterize glutamate transporter function.

In some cases, a membrane transporter assay described herein (e.g., OSTA) can be used to identify a substrate for a membrane transporter. A method of identifying a membrane transporter substrate can include expressing a sensor in a cell having a membrane transporter, perfusing the cell with a solution having a candidate substrate, and detecting the candidate substrate. During the perfusion, the substrate solution can oscillate between having a low substrate concentration and having a high substrate concentration. Detection of the candidate substrate can indicate that the candidate substrate is a substrate for the membrane transporter. Failure to detect the candidate substrate can indicate that the candidate substrate is not a substrate for the membrane transporter.

In some cases, a membrane transporter assay described herein can be used to screen for membrane transporter modulators (e.g., agonists or antagonists). For example, OSTA can be used to screen for agonists of membrane transporter function and/or antagonists of membrane transporter function. A membrane transporter modulator can modulate a membrane transporter involved in a disease and/or disorder. A modulator of any appropriate membrane transporter involved in a disease and/or disorder can be assayed using a membrane transporter assay described herein. Examples of diseases and/or disorders associated with membrane transport include, without limitation, cystic fibrosis, cerebral stroke, epilepsy, Alzheimer's disease, Huntington's disease, and amyotrophic lateral sclerosis (ALS). Examples of membrane transporters involved in a disease and/or disorder include, without limitation, SLC26a3, EAAT2, and NBCe1-b. A method of screening for a membrane transporter modulator can include expressing a sensor in a cell having a membrane transporter, perfusing the cell with a solution having substrate transported by the membrane transporter, providing the cell with a candidate modulator, and detecting the substrate. During the perfusion, the substrate solution can oscillate between having a low substrate concentration and having a high substrate concentration. Detection of a change in transport of the substrate (e.g., compared to transport of the substrate in the absence of the candidate modulator) can indicate that the candidate modulator is a modulator for the membrane transporter. For example, an increase in membrane transport function can indicate that the candidate modulator is an agonist for the membrane transporter. For example, a decrease in membrane transport function can indicate that the candidate modulator is an antagonist for the membrane transporter. Detection of the substrate transport can indicate that the candidate modulator is not a modulator for the membrane transporter. In some cases, a modulator of membrane transporter function can be administered to a cell to modulate membrane transporter function. For example, a modulator of membrane transporter function can be administered to a cell to treat (e.g., to reduce the symptoms of) a membrane transporter associated disease or disorder.

The invention will be further described in the following examples, which do not limit the scope of the invention described in the claims.

### EXAMPLES

Current transporter assays rely on single bouts of activity: substrate is added or removed, and resulting changes over time are recorded. In OSTA, however, continuous perfusion provides the ability to add or remove substrate reliably and repeatedly, and the number and rapidity of measurement events is limited only by the combined properties of the experimental setup. OSTA was used to evaluate a wide variety of transporter properties.

### Materials and Methods

### OSTA

A schematic representation of an OSTA is shown in FIG. 1. Cells expressing the transporter of interest were loaded with either a small molecule or a genetically-encoded sensor and were perfused with solutions oscillating between high and low substrate concentrations. Data quality was improved by tailoring experimental parameters to produce a triangular waveform that never reaches equilibrium. The slope of this readout thus becomes a nearly constant monitor of transport rate (FIG. 2).

### Cell Culture

All experiments presented were performed on HEK-293 (ATCC #CRL-1573) cells transfected using the Amaxa® system (Lonza). Cells were cultured after transfection to densities of 50-90% confluence in 35 mm coverslip-bottomed culture dishes (MatTek), either uncoated or pre-coated with fibronectin (Sigma-Aldrich), and were imaged in situ. Effects of coating on cell adherence were not quantified, but uncoated dishes worked sufficiently well to allow measurements in most cases.

### Sensors and Dyes

For pH imaging, 50 μg aliquots of SNARF-5F-AM (ThermoFisher S23923) were dissolved in dimethyl sulfoxide (DMSO) to 20 mM, diluted 1:200 (loading concentration 100 μM) in existing culture medium from dish, and cells were incubated for 20-30 minutes under time-lapse imaging (1 frame per 10 seconds) to determine completeness of loading. Cells loaded in this manner provided data for more than 1 hour under constant imaging, although intensities decayed moderately over time. The ratiometric nature of the dye, however, compensated for decay-related artifacts. For glucose imaging, a cytosolic ratiometric genetically encoded glucose sensor (Keller et al, manuscript in preparation) was co-transfected with Sglt2. For glutamate imaging, a version of iGluSnFR (Marvin et al., 2013 Nat. Methods 10:162-170) was used in which the signal peptide and transmembrane domains were removed, thus targeting the sensor to the cytosol.

### Perfusion System

An off-the-shelf, gravity-fed, four-channel perfusion system (VC3-4, ALA instruments) was used in all experiments. The bottoms of the 60 mL feeder syringes were aligned vertically to 1-2 cm below the microscope stage (outlet level), which provided flow rates of 4-8 mL/minute depending on the fluid level of the syringes. Buffers were gassed by continuous bubbling with 5% CO₂ when appropriate. The outlet of the system was directed towards the illuminated area of the coverslip dish at a distance of 3-5 mm, and continuous fast suction at the raised edge of the dish removed solutions. The perfusion outlet was Tygon tubing with 1/16″ inner diameter (somewhat larger than that in the manifold), which might have slowed the velocity of the buffers, allowing for better cell adhesion. Protocols for buffer switching were carried out by the software provided. Buffer changes were characterized to be >90% in 1 second and ˜100% in 1.5 seconds (FIG. 3).

### Imaging System

All imaging was performed on an LSM-510 (Zeiss) inverted confocal fluorescence microscope, with excitation from argon laser lines at 488 and/or 514 nm, or DPSS at 561 nm. Objectives were Zeiss EC Plan-Neofluar 10×/0.3 NA M27 and Plan-Apochromat 20×/0.8 NA. Separate experiments indicated that conventional epifluorescence microscopes provided similar results. Imaging was carried out at fastest frame rate possible (2 Hz) with bi-directional scanning and pixel dimensions of 512×512 with either 8- or 16-bit image depth. The lower-magnification objective was preferred due to its inclusion of more cells, and although higher magnification always improved signal-to-noise, larger numbers of cells were considered preferable for population sampling. Pinholes were set to their maxima, corresponding to z-depths of 126 μm and 25 μm for the 10× and 20× objectives, respectively, in order to lessen potential motion artifacts in the z dimension. Laser power at 488 and 514 nm was generally set to 100% and nevertheless did not cause noticeable photobleaching, probably because of the fast frame rates, low magnification, and weakness of the aging laser. In the case of the Sglt2 experiment, the DPSS laser was set to 2-10% in the attempt to limit photobleaching of the mRuby2 moiety of the glucose sensor, but even at 2% power photobleaching was unavoidable due to a combination of mRuby2's relative photosensitivity as well as its susceptibility to photobleaching from the 488 nm laser used concurrently. The artifact, while noticeable, did not affect conclusions. Detector gains were always set to prevent overloads of pixels.

### Example 1

### SLC26a3-Mediated Transport of Cl⁻/HCO₃⁻

SLC26a3 (A3), a Cl⁻/HCO₃⁻ exchanger, is expressed in many tissues where it plays various roles in pH regulation and chloride balance (Ohana et al., 2009 J. Physiol. 587:2179-2185). A3 exchange of Cl⁻/HCO₃⁻ was characterized with the OSTA method (FIG. 4). Cells expressing A3 were loaded with the pH-sensitive dye SNARF-5F-AM, and perfused with buffers oscillating every 5 s (total period 10 s) between 8 and 158 mM Cl⁻, and in the presence of 25 mM HCO₃⁻, and with a confocal microscope frame rate of ˜2 Hz (FIG. 4A). A uniform oscillating response in the SNARF-5F signal was observed to follow the stimulus (FIG. 4B-D). Since the drug salicylate is known to inhibit a nontransporting paralog to A3, prestin (SLC26a5) (Santos-Sacchi et al., 2006 J. Neurosci. 26:3992-3998; Zheng et al., 2000 Nature 405:149-155), 10 mM salicylate was added to the oscillating buffers to determine its effect on A3. At this concentration, salicylate was found to abrogate nearly completely the oscillating response, and subsequent washout showed a return to the initial oscillating response. Further studies are underway to quantify the affinity (IC₅₀) and to explore physiological implications of this interaction. In an experiment of 15 minutes' duration, not only was the OSTA paradigm demonstrated and previous results confirmed, but a novel hypothesis was also tested and confirmed, at least to a first approximation.

### Example 2

### SLC26a2-Mediated Exchange of SO₄²⁻/OH⁻

Another transporter from the SLC26 family, SLC26a2 (A2), has been implicated in a number of osteochondrodysplasias (bone growth abnormalities) due to mutations affecting its normal SO₄²⁻/OH⁻ exchange activity (Dawson and Markovich, 2005 Curr. Med. Chem. 12:385-396), which is critical for sulfation of cartilage and extracellular matrix. Normal transport has been reported to require extracellular Cl⁻ to allosterically gate the normal SO₄²⁻/OH⁻ exchange function (Ohana et al., 2012 J. Biol. Chem. 287:5122-5132). Thus, an experiment was devised in which permutations of SO₄²⁻, Cl⁻, and OH⁻ could be perfused onto SNARF-5F-loaded cells expressing A2 (FIG. 5A). The primary stimulus was oscillation of SO₄²⁻ from 0 to 100 mM with a period of 40 seconds, and this was continued throughout the entire experiment. In the first phase of the experiment (FIG. 5B-D), the buffers were held at pH 7.5 and 0 Cl⁻. As expected, this resulted in negligible oscillations in transport signal due to lack of extracellular Cl⁻, but when 10 mM Cl⁻ was superadded to these solutions, oscillations became apparent, consistent with the reported allosteric necessity of Cl⁻ for transport. The same regimen was repeated at pH 6.85, with greatly diminished but still marked oscillation amplitudes in the Cl⁻-containing condition, and at pH 8.5, which replicated the pH 7.5 condition. This lack of difference between pH 7.5 and 8.5 was unexpected, since the concentration of the transporter's reported substrate, OH⁻, increases 10-fold from pH 7.5 to pH 8.5. The unexpected result can perhaps be attributed to saturation of the OH⁻ binding site, but was not explored further.

It was noted that the average “tonic” intracellular pH in the absence of extracellular chloride was always lower in A2-expressing cells than in controls. This was consistent with claims that extracellular chloride is required only for influx of OH⁻, and not efflux (Ohana et al., 2012 J. Biol. Chem. 287:5122-5132). The transport cycle in the absence of Cl⁻ would thus favor efflux of OH⁻ and would lower intracellular pH, as observed herein. The inverse finding, however, was also observed: in the presence of extracellular chloride, tonic intracellular pH was higher in A2-expressing cells than in controls. One possible explanation is that Cl⁻ not only gated OH⁻ entry, but also dictated asymmetry in the transport process, perhaps by allosterically biasing the transporter towards a conformation favorable to OH⁻ influx. In any case, this experiment demonstrates the ease with which transporter function under various permutations of conditions can be quickly yet rigorously tested by the OSTA method.

### Example 3

### Sglt2-Mediated Transport of Na⁺/Glucose

To explore the OSTA's efficacy on transporters of more diverse types, experiments were designed to test the diabetes-related drug target Sglt2. This Na⁺/glucose cotransporter is expressed physiologically in the kidney, where, driven by sodium gradients, it recovers the majority of the glucose initially filtered into the urine (Wright, 2001 Am. J. Physiol. Renal Physiol. 280:F10-18). Potent and specific inhibitors have been developed, with the rationale that significant amounts of glucose could be removed from the circulation by blocking recovery and allowing it to be excreted from the body in urine. Some of these inhibitors are clinically approved and in use (Meng et al., 2008 J. Med. Chem. 51:1145-1149). Unfortunately, Sglt2 tends to have a low flux rate in various expression systems (Wright, 2001 Am. J. Physiol. Renal Physiol. 280:F10-18), and is therefore generally difficult to measure in conventional transport assays. To test the measurability of Sglt2's transport under the OSTA paradigm, cells were co-transfected with both Sglt2 and a novel intracellular ratiometric fluorescent glucose sensor protein (Keller et al, manuscript in preparation), and subjected to oscillations in extracellular glucose in the presence of Na⁺. Because control cells without Sglt2 transfection showed significant background glucose oscillations due to endogenous transport, conditions were optimized to allow for a greater prominence of specific Sglt2 signal. Oscillations of Na⁺/K⁺ were superimposed either in conjunction or in opposition to the glucose oscillations, shifting simultaneously both the co-substrate (Na⁻) gradient as well as the transmembrane potential (K⁺) driving Sglt2's electrogenic transport (FIG. 6A). Using this paradigm, Sglt2-mediated glucose fluxes (FIG. 6B-D) became distinguishable from transmembrane potential- and Na⁺-insensitive background oscillations as intervals of increased amplitude when all gradients favored Sglt2-mediated transport. To ensure that this was Sglt2-specific and not an artifact induced by variations in buffer contents, the Sglt2 inhibitor dapagliflozin (EC₅₀ 1.1 nM (Meng et al., 2008 J. Med. Chem. 51:1145-1149)) was added at 500 nM. Dapagliflozin quickly and completely abrogated the regions of heightened amplitude identifying Sglt2 as the cause thereof. OSTA was shown to work in another system of perhaps more difficult accessibility.

### Example 4

### EAAT2-Mediated Transport of Glutamate

Due to its prominence and its intricate stoichiometry (3 Na⁺, 1 H⁺, and 1 glutamate for 1 K⁺ (Levy et al., 1998 J. Neurosci. 18:9620-9628)), EAAT2 seemed another appropriate candidate through which to explore further the universality of the method. Excitatory amino acid transporters (EAAT's) were first described in 1978 (Kanner and Sharon, 1978 Biochemistry 17:3949-3953), and have since become well known for their critical role at glutamatergic synapses in the central nervous system (CNS), where they re-uptake the neurotransmitter glutamate and thereby terminate signaling and prevent excitotoxicity (Jensen et al., 2015 Curr. Opin. Pharm. 20:116-123; Nakagawa and Kaneko, 2013 Curr. Mol. Pharmacol. 6:66-73). EAAT2, also known as GLT-1 or SLC1A2, is primarily expressed in astrocytes, and is responsible for the majority of glutamate reuptake in the CNS (Tanaka et al., 1997 Science 276:1699-1702). Defects in EAAT2 are implicated in cerebral stroke, epilepsy, Alzheimer's disease, HIV-associated dementia, Huntington's disease, amyotrophic lateral sclerosis (ALS) and malignant glioma (Shigeri et al., 2004 Brain Res. Rev. 45:250-265), as well as traumatic brain injury (Yi and Hazell, 2006 Neurochem. Int. 48:394-403). EAAT2 is, therefore, a key drug target for these conditions as well as other excitotoxic states.

To evaluate OSTA's efficacy in measuring EAAT-mediated transport, cells were co-transfected with EAAT2 and a cytosolic version of the glutamate sensor iGluSnFR (Marvin et al., 2013 Nat. Methods 10:162-170), and buffers were oscillated between 0 and 10 mM glutamate. This protocol did not produce significant signals. This was likely due to basal levels of intracellular glutamate saturating the sensor, whose K_{d} is 80 μM (Marvin et al., 2013 Nat. Methods 10:162-170). This sensor-saturation hypothesis was corroborated by depriving EAAT2/iGluSnFR-expressing cells of extracellular glutamate overnight: when glutamate was restored, dF/F rose to a maximum of ˜2.5 (the maximum when recorded on the surface of HEK cells was ˜4-fold (Marvin et al., 2013 Nat. Methods 10:162-170)), remained high in the absence of glutamate, and was unresponsive to subsequent glutamate pulses (FIG. 8). Iit appeared that cells, or at least those used herein, had a homeostatic mechanism to maintain cytosolic glutamate concentrations above the high micromolar range. The saturation effect might in future experiments be obviated by tuning the sensor to a weaker affinity through targeted mutations to the ligand binding site or the hinge region (Marvin and Hellinga, 2001 Nat. Struct. Biol. 8:795-798), some of which have already been described (Marvin et al., 2013 Nat. Methods 10:162-170). Another potentially confounding aspect of these measurements is EAAT2's co-transport of H+, which would tend to decrease the sensor's brightness (iGluSnFR is moderately pH-sensitive (Marvin et al., 2013 Nat. Methods 10:162-170)), in opposition to the increases engendered by glutamate. Indeed, several cells showed weak responses of opposite phase to those expected from glutamate, suggesting a H⁺-mediated quenching effect on the saturated sensor. Nevertheless, based on pH titrations of the sensor and plausible intracellular pH values, the effect was small in comparison to glutamate-driven oscillations from an unsaturated sensor. These issues are presented to demonstrate potential hurdles in implementation of OSTA; they did not, however, prevent success in any transporter targeted in this study or otherwise.

To overcome the obstacles above, cells were exposed to oscillations of extracellular K⁺ (150 mM, Natfree) of phase opposite that of Na⁺/glutamate. Through this combined Na⁺/K⁺/glutamate stimulus, EAAT2 was apparently induced to run in reverse and export glutamate, lowering intracellular glutamate levels enough to de saturate the sensor and unmask a robust oscillating response of appropriate phase in a subset of iGluSnFR-expressing cells. This signal was then harnessed to test the effects of two compounds. Addition of 2 μM of the EAAT2 inhibitor TFB-TBOA completely abolished the oscillations, with rapid onset and slower washout, consistent with its reported high affinity (IC50˜17 nM (Tsukada et al., 2005 Neuropharmacology 48:479-491)). As a negative control, TFB-TBOA was removed and sodium salicylate (10 mM) was added under the same stimulus, with no effect on EAAT2 activity besides a small tonic baseline decrease, probably due to salicylate's activity as a protonophore (Saeedi et al., 2013 J. Exp. Bot. 64:1829-1836). An equivalent tonic shift was seen in untransfected cells, indicating that it was EAAT2-independent (FIG. 7B). Taken together, these results show that OSTA can be used with a variety of transporters to explore transporter function quickly and easily.

### Example 5

### Intracellular Organelle Glucose Transport: Endoplasmic Reticulum (ER)

Experimental conditions were devised to allow measurement of transport in intracellular organelles. There were two main obstacles to attaining this goal: 1. specific localization of the sensor to the organelle of choice, and 2. rapid control of intracellular substrate concentrations. Glucose transport in the ER was selected since previous work has already shown glucose fluxes occur in the ER (Fehr et al., 2005 Mol. Cell. Biol. 25, 11102-11112), and since an ER-targeted version of the glucose sensor (modified from the sensor used in Example 3; manuscript in preparation) was available in the laboratory. There are numerous well-characterized protein localization motifs, and this therefore represents a general strategy for targeting protein-based sensors to the organelle of choice, and some similar methods exist for dye-based sensors, e.g., targeting pH indicator dyes to organelles (Benink et al., 2009 BioTechniques 47, 769-774). The second issue, manipulation of intracellular substrate concentration (glucose in this case), was overcome by screening six different glucose transporters for their rapidity of glucose transport into the cytosol, as measured using OSTA and the cytosolic version of the glucose sensor (˜3 hours total imaging). It was determined that Glut1 was by far the fastest under the experimental conditions tested, with some cells exhibiting nearly square-wave response. Glut1 expression permeablized the cell membrane to glucose, enabling oscillating glucose stimuli to reach the ER.

To apply OSTA to ER glucose transport, parallel batches of cells were co-transfected with Glut1 and either the cytosolic or the ER-localized sensor, and OSTA was applied under identical conditions (including solutions, imaging, and processing). Responses were large, fast, and saturating for the cytosolic sensor, but were much smaller, slower, and almost completely linear for the ER-localized sensor (FIG. 5). The linearity of the ER transport signal suggests that ER-based transport is rate-limiting, and is not complicated by insufficient rapidity in the cytosolic glucose oscillations; this linear signal is therefore a direct and constant temporal readout of glucose transport across the ER membrane. This experiment demonstrates that it is possible, with appropriate experimental design, to use OSTA to measure transporter function even in organelles. It will be of significant interest to determine rates of transport in situ in other types of organelles, in different cell types, under various conditions, or including various pharmacological agents.

### Example 6

### Temperature and Glucose Transport

Since OSTA uses constant fast perfusion, temperature-controlled experiments can be carried out using temperature-controlled perfusion alone. To test this, the glucose transport of Glut2, as monitored by the cytosolic version of the same glucose sensor as above, was measured at 26 and 37° C. using OSTA. When the perfusate temperature was changed from 26 to 37° C., an approximately three-fold change was observed in the magnitude of transport rates (FIG. 5D), demonstrating an effect of temperature of roughly the same magnitude as in previous studies, e.g., hexose transport in hepatoma cells changed ˜3-4.5-fold over this temperature change (Plagemann et al., 1981 Biochemistry 20:3366-3370) or ˜3.5-4 in red blood cells (Hankin and Stein, 1972 Biochimica et biophysica acta 288:127-136). This effect was determined not to be an artifact of temperature-dependence of the sensor (FIG. 8). Although temperature control in the current experiments was achieved simply by running extra lengths of perfusion tubing through a water bath of known temperature, use of a more sophisticated, commercially-available temperature-control apparatus would provide means to measure temperature-dependent aspects of transporter function quite readily and precisely, perhaps in conjunction with inhibitors or other experimental manipulations described above, to procure detailed mechanistic information about various transport processes.

### Example 7

### Fourier Transforms

### Frequency Domain Analysis of OSTA Datasets

In the analysis stage of the above experiments, finding appropriate ROIs (segmentation) was sometimes difficult. One method was to transfect the transporter of interest fused genetically to a fluorescent reporter protein, but this may have perturbed transporter function and surprisingly did not provide ideal ROIs. This is due to both weak visibility of the reporter due to membrane localization as well as “hot spots” due to non-functional ER-accumulated protein: reporter intensity correlated poorly with functional signal. Another approach taken was to co-transfect a separate cytosolic reporter protein plasmid, but it was observed that cells often seemed to receive only one of the two plasmids, again leading to poor reporter-function correlation. Upon visual inspection of the image stacks, however, time-dependent oscillating functional signatures were visible to the eye. It was realized that these temporal signatures could be computationally identified through Fourier transforms (FTs) of each pixel in the time dimension: pixels with the greatest oscillations would be highlighted in the FT at the stimulus frequency. Since the frequency of the stimulus was known, and the response was presumably of identical frequency, the appropriate images in the Fourier-transformed image stacks (both phase and amplitude) could be selected and used for segmentation of ROIs. This approach is called Fourier Transform OSTA (FT-OSTA).

As a proof-of-principle of this method, data similar to the initial segment of FIG. 4 were analyzed. First, pixel-wise temporal fast Fourier transforms (FFTs) of the image stack were computed using a plugin to ImageJ (see FIG. 9 for plugin workflow schematic). This plugin outputs a stack of images with its time domain transformed into frequency domain, with interleaved real and imaginary images; as such, each image represents the real or imaginary response component of all pixels at a given frequency. This stack can be further transformed with separate plugins to amplitudes or phases, again with each image representing a particular frequency. For these data, the signal was well isolated in two successive images in the frequency domain stacks (FIG. 10). These images were then used for the definition of ROIs and measurement of functional data from the original time-domain stack. The functional traces derived from these ROIs displayed much higher signal-to-noise ratios than those derived from other means of segmentation (based on fluorescent co-transfection markers or tagging).

### FT-OSTA Analysis in the Presence of Artificial or Experimental Noise

To benchmark FT-OSTA, substantial artificial Gaussian noise (pixel-wise standard deviations increased ˜10-fold) was added to the same dataset, and results were compared (FIG. 11). On a visual level, the original images had shown clear cell boundaries and functional signatures, but these disappeared entirely upon addition of noise unless the contrast was carefully adjusted to the original bounds, in which case the cells became barely detectable (FIG. 11A, E). Using only the raw data, definition of ROIs would have been impossible. FT-OSTA analyses then carried out on both the original and noisy image stacks. A further step was taken this time; the phase and amplitude information were combined, thus recapitulating a lock-in amplifier and thereby boosting the signal. To establish a metric for signal quantification in the resultant images, a large square ROI was defined over a non-responsive region (outlined in FIG. 11H) and its standard deviation was measured for each of the stimulus-frequency FT images. Images were then divided by this standard deviation to provide images calibrated in units of standard deviations (sigma (a)) above background. The original dataset showed a large signal, with some cells exhibiting signals greater than 50 σ in the amplitude images and greater than 125 σ in the hybrid phase-amplitude images (FIG. 11B-D). As expected, the signal in the noisy dataset was significantly attenuated, but was nevertheless readily detectable, with some cells reaching levels of ˜25 σ (FIG. 11FH). The level of noise in this exercise, as can be seen in one example ROI (FIG. 11I-J), is probably such to preclude meaningful measurements in the time domain: while a signal's presence is obvious, its quantity is poorly defined without further processing. It is likely, although not explored in the current study, that a combined time-frequency analysis approach via FT analysis of sliding temporal “windows” of images could be used to measure oscillating signals as a function of time, especially with signals of high temporal frequency. (An analogous analysis is in fact done biologically in the auditory system with sounds, where it seems to be highly effective (Gabor, 1946 J. Inst. Elect. Eng. III:429-457).) In any case, it can be seen from this example that even when the cells themselves become invisible to conventional microscopy, FT-OSTA methods can reveal signals from responsive cells.

### Example 8

### NBCe1-b-Mediated Transport of Na+/HCO3-

To test the method in an actual experimental scenario, a complex and experimentally noisy dataset was re-analyzed using FT-OSTA. Using conventional methods, this dataset appeared to be too noisy to provide definitive information, but by finding the most responsive ROIs through FT-OSTA methods, functional traces were improved enough to allow conclusions to be drawn. The subject of this experiment was the Na⁺/HCO₃⁻ electrogenic co-transporter NBCe1-b, which has a variable stoichiometry of 1 Na⁺: 2 or 3 HCO₃⁻ (Romero et al., 2013 Mol. Aspects Med. 34:159-182). NBCe1-b is critical in renal and ocular physiology, and its mutations have been connected to diseases of these organs (Romero et al., 2013 Mol. Aspects Med. 34:159-182). Since the transporter had previously been found to be electrogenic, altering transmembrane potential by manipulating extracellular K⁺ concentrations should augment or diminish transport when in conflict or in concert with Na⁺-driven fluxes. Accordingly, permutations of Na⁺ and K⁺ (FIG. 12A) were used to query the transporter for electrogenicity and Na⁺ dependence (FIG. 12B). Since this transporter responded far more slowly than the others tested in this study, longer periods (120 seconds) had to be used to achieve measureable signals, and even so, baselines drifted and the response magnitudes were comparatively tiny. Therefore, several image processing techniques were used. First, FT amplitude analyses were carried out on the raw images to find both high-amplitude (transporter-positive) and low-amplitude (control) baseline ROIs. Next, to remove the slowly drifting non-periodic baseline, a moving-window-average subtraction plugin was used with a window size matched to the stimulus period. With arrangement, the plugin subtracts a non-oscillating, a smoothly-varying baseline (FIG. 12C, gray trace) which should not affect the oscillating components. Traces from ROIs were then averaged, and the resultant trace from low-amplitude ROIs was subtracted from that of the high-amplitude ROIs, to remove other systematic artifacts. In the end, the FT-OSTA procedures yielded a clean trace (FIG. 12C, black trace) that varied under different conditions in ways predicted from the reported stoichiometry, as follows.

In the first stage (I) of the experiment (for this section, refer to FIG. 12A-D), K⁺ was oscillated in the absence of Na⁺ to change the transmembrane voltage, and to confirm NBCe1-b's requirement of Na⁺ (Romero et al., 2013 Mol. Aspects Med. 34:159-182). For unknown reasons, relatively small-amplitude oscillations did occur, perhaps because of residual Na⁺, or perhaps Na⁺ is not absolutely required. In the second stage of the experiment (II), Na⁺ concentrations were oscillated while keeping the relatively low K⁺ concentration unchanged, resulting in the expected oscillations in pH. In the third stage (III), both oscillating gradients (Na⁺ and K⁺-driven transmembrane potential) were applied in conjunction, resulting in still larger oscillations in pH. In the next stage (IV), these same oscillations were set in opposition (anti-phase), which diminished the oscillations to a magnitude smaller than the single Na⁺ gradient. In the next stage (V), Na⁺ oscillations alone were again used to drive transport, but this time at a depolarized potential through increased K⁺ concentration. The oscillations did not differ significantly from those at lower K⁺ concentrations (stage II), suggesting that tonic transmembrane potential does not affect transporter efficiency through hypothetical voltage-gating or otherwise. Finally, in the last stage (VI), the effects of transmembrane potential oscillations alone were tested, and were found to be smaller than Na⁺-driven oscillations, but larger than the background oscillations in (I). These findings were in accordance with previously reported results, but were measured in a single experiment testing both Na⁺ dependence as well as electrogenicity. It should be noted that the demands of electrogenicity experiments like this one preclude measurements of transporters whose transport cycle involves K⁺ ions, e.g., EAAT2, and potentially those involving Cl⁻, since cells often have non-trivial Cl⁻ conductances, thus confounding the effects of extracellular K⁺ on transmembrane potential. In all transporters indifferent to these ions, however, such measurements should be feasible. Although NBCe1-b displayed the weakest response by far of all transporters tested, and might represent a worst-case scenario, the combination of experimental and analytical techniques described above (FT-OSTA) was able to extract data in accordance with previously reported transport properties.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A method of characterizing membrane transporter function, said method comprising:
a) expressing a sensor in a cell comprising a membrane transporter, wherein the sensor capable of detecting a substrate transported by the membrane transporter;
b) perfusing the cell with a solution comprising the substrate, wherein said solution changes or oscillates between a low substrate concentration and a high substrate concentration; and
c) detecting the substrate, wherein changes in detection of the substrate can be used to characterize membrane transporter function.

2. The method of claim 1, wherein the membrane transporter is an exogenous membrane transporter.

3. The method of claim 1, wherein the membrane transporter is selected from the group consisting of glucose transporter 1 (GLUT1), GLUT2, solute carrier family 26 member 3 (SLC26a3), SLC26a2, Sglt2, EAAT2, and NBCe1-b.

4. The method of claim 3, wherein the membrane transporter is SLC26a3, and wherein the membrane transporter substrate comprises Cl⁻ and HCO₃⁻.

5. The method of claim 3, wherein the membrane transporter is SLC26a2, and wherein the membrane transporter substrate comprises SO₄²⁻ and OH⁻.

6. The method of claim 3, wherein the membrane transporter is Sglt2, and wherein the membrane transporter substrate comprises Na⁺ and glucose.

7. The method of claim 3, wherein the membrane transporter is EAAT2, and wherein the membrane transporter substrate comprises glutamate.

8. The method of claim 3, wherein the membrane transporter is NBCe1-b, and wherein the membrane transporter substrate comprises Na⁺ and HCO₃⁻.

9. The method of claim 1, wherein the expressing a sensor comprises transfecting the cell with a nucleotide sequence encoding a peptide sensor.

10. The method of claim 1, wherein the sensor is a fluorescent sensor.

11. The method of claim 10, wherein the fluorescent sensor is a fluorescent Ca²⁺ sensor.

12. The method of claim 11, wherein the fluorescent Ca²⁺ sensor is selected from the group consisting of Furas, OGB, Fluos, and Indos.

13. The method of claim 10, wherein the fluorescent sensor is a fluorescent Mg²⁺ sensor.

14. The method of claim 13, wherein the fluorescent Mg²⁺ sensor is selected from the group consisting of mag-Indo, mag-Fura, and mag-Fluo.

15. The method of claim 10, wherein the fluorescent sensor is a fluorescent voltage sensor.

16. The method of claim 15, wherein the fluorescent voltage sensor is a di-ANNEPSs or a PeTs.

17. The method of claim 10, wherein the fluorescent sensor is a fluorescent H⁻ sensor.

18. The method of claim 17, wherein the fluorescent H⁺ sensor is a SNARF dye or a BCECF dye.

19. The method of claim 18, wherein the fluorescent H⁺ sensor is a SNARF dye, and wherein said SNARF dye is a SNARF-5F-AM.

20. The method of claim 10, wherein the fluorescent sensor is a fluorescent glucose sensor.

21. The method of claim 20, wherein the fluorescent glucose sensor is a cytosolic ratiometric peptide.

22. The method of claim 10, wherein the fluorescent sensor is a fluorescent glutamate sensor.

23. The method of claim 22, wherein the fluorescent glutamate sensor is iGluSnFR or a sensor for tryptophan, nicotine, alanine, glycine, proline, maltose, maltotriose, pH, or ATP.

24. The method of claim 1, wherein the cell is a mammalian cell.

25. The method of claim 24, wherein the mammalian cell is an adherent cell.

26. The method of claim 24, wherein the mammalian cell is a HEK-293 cell.

27. The method of claim 1, wherein the perfusing is continuous perfusion.

28. The method of claim 1, wherein the oscillation period is about 10-120 seconds.

29. The method of claim 1, wherein the solution oscillates between about 0-160 mM substrate concentration.

30. The method of claim 1, wherein the detecting the substrate comprises obtaining one or more images of the cell.

31. The method of claim 30, wherein a series of images of the cell are obtained using a microscope.

32. The method of claim 31, wherein the sensor is a fluorescent sensor, and wherein the microscope is a fluorescence microscope.

33. The method of claim 31, wherein the series of images of the cell is a time-lapse movie.

34. The method of claim 33, wherein the time-lapse movie comprises frame rates of 1-2 Hz.

35. The method of claim 1, wherein the membrane transporter function that is characterized is selected from the group consisting of transport rate, transporter activity, transporter and inhibition.

36. A method of characterizing Cl⁻/HCO₃⁻ exchange across a cell membrane, said method comprising:
a) expressing a fluorescent H⁺ sensor in a cell comprising a Cl⁻/HCO₃⁻ exchange transporter, wherein the fluorescent H⁺ sensor comprises a SNARF-5F-AM dye;
b) perfusing the cell with a solution comprising Cl⁻, wherein said solution oscillates between about 8 mM Cl⁻ and about 158 mM Cl⁻; and
c) detecting the H⁺, wherein changes in the H⁺ can be used to characterize Cl⁻/HCO₃⁻ exchange across a cell membrane.

37. A method of characterizing SO₄²⁻/OH⁻ exchange, said method comprising:
a) expressing a fluorescent H⁺ sensor in a cell comprising a SO₄²⁺/OH⁻ exchange transporter, wherein the fluorescent H⁺ sensor comprises a SNARF-5F-AM dye;
b) perfusing the cell with a solution comprising SO₄²⁻, wherein said solution oscillates between about 0 mM SO₄²⁻ and about 100 mM SO₄²⁻; and
c) detecting H⁻, wherein changes in the H⁺ can be used to characterize SO₄^{2'1}/OH⁻ exchange.

38. A method of characterizing glucose transporter function, said method comprising:
a) expressing a fluorescent glucose sensor in a cell comprising a glucose transporter, wherein the fluorescent glucose sensor is a cytosolic ratiometric peptide;
b) perfusing the cell with a solution comprising glucose, wherein said solution oscillates between about 0 mM glucose and about 2 mM glucose; and
c) detecting the glucose, wherein changes in detection of the glucose can be used to characterize glucose transporter function.

39. The method of claim 38, wherein the glucose transporter is a Na⁺-dependent glucose transporter function, and wherein the perfusing step further comprises perfusing the cell with a solution comprising Na⁺.

40. A method of characterizing glutamate transporter function, said method comprising:
a) expressing a fluorescent glutamate sensor in a cell comprising a glutamate transporter, wherein the fluorescent glutamate sensor comprises iGluSnFR;
b) perfusing the cell with a solution comprising glutamate, wherein said solution oscillates between about 0 mM glutamate and about 10 mM glutamate; and
c) detecting the glutamate, wherein changes in detection of the glutamate can be used to characterize glutamate transporter function.

41. A method of identifying a substrate of a membrane transporter, said method comprising:
a) expressing a sensor in a cell comprising a membrane transporter, wherein the sensor can detect a candidate substrate;
b) perfusing the cell with a solution comprising the candidate substrate, wherein said solution oscillates between a high candidate substrate concentration and a low candidate substrate concentration; and
c) detecting transport of the candidate substrate, wherein transport of the candidate substrate identifies the candidate substrate as the substrate of the membrane transporter.

42. A method of screening for modulators of membrane transporter function, said method comprising:
a) expressing a sensor in a cell comprising a membrane transporter, wherein the sensor can detect a substrate transported by the membrane transporter;
b) perfusing the cell with a solution comprising the substrate, wherein said solution oscillates between a low substrate concentration and a high substrate concentration;
c) providing the cell with a candidate modulator; and
d) detecting transport of the substrate, wherein a change in transport of the substrate identifies the candidate modulator as a modulator of membrane transporter function.
